(19) United States
(12) Patent Application Publication
(10) Pub. No.: US 2011/0269220 A1
(43) Pub. Date: Nov. 3, 2011

(54) GORDONIA SIHWENSIS AND USES THEREOF

(75) Inventor: Donald C. Van Slyke, (US)

(73) Assignee: CHEVRON U.S.A. INC., San Ramon, CA (US)

(21) Appl. No.: 13/168,793

(22) Filed: Jun. 24, 2011

Related U.S. Application Data
(63) Continuation-in-part of application No. 12/338,581, filed on Dec. 18, 2008.

(51) Int. Cl. C02F 3/34; A62D 3/02; C12N 1/20

(52) U.S. Cl. 435/262; 435/252.1; 210/601

(57) ABSTRACT

Strains of Gordonia sihwensis and uses thereof are described herein. G. sihwensis can sequester and/or biodegrade hydrocarbons. In particular, G. sihwensis may be used in remediation of drill cuttings coated with drilling fluid and soil or sludges contaminated with oil contaminants.

### CROSS REFERENCE TO RELATED APPLICATIONS

The present application is a continuation-in-part of U.S. application Ser. No. 12/338,581, filed Dec. 18, 2008, the entirety of which is hereby incorporated by reference.

### 1. FIELD OF THE INVENTION

Gordonia sihwensis described herein may be used to sequester and/or biodegrade hydrocarbons. In particular, Gordonia sihwensis described herein may be used in the remediation of drill cuttings coated with drilling fluid.

### 2. BACKGROUND

Environmental pollution with hydrocarbons poses a major concern. Crude oil is a major sea pollutant, and petroleum products, such as gasoline and diesel fuel and fuel oils, are the most frequent organic pollutants of soils and ground waters. In the drilling of oil and gas wells, oil-based drilling fluid is required in most of the challenging drilling situations, and the spent oil-coated drill cuttings cannot typically be discharged from the drilling rig for environmental reasons. A rapid biodegradation of oil on such cuttings could render oil-based drilling fluids as environmentally acceptable as water-based drilling fluids.

There are two primary types of drilling fluids: (i) water based drilling fluids (WBF); and (ii) non-aqueous drilling fluids (NADFs). WBFs comprise water mixed with bentonite clay and barite to control mud density and thus, hydrostatic head. Other substances can be added to affect one or more desired drilling properties. NADFs are either based on mineral oil, diesel, or synthetic base fluid. NADFs are typically water in oil (invert) emulsions. In rare cases, such as with coring fluids, 100% oil-based drilling fluids have been used. NADFs are generally preferred over water-based fluids for their ability to provide superior borehole stability, lubricity, rate of penetration, stuck pipe prevention, chemical stability, and corrosion protection.

In contrast to WBFs and WBF-coated cuttings that can typically be discharged into the environment, in many areas regulatory standards do not allow the discharge of NADFs, or drill cuttings coated with NADF into the environment. If NADF-coated cuttings are not permitted to be discharged into the environment, then the cuttings must either be reinjected, hauled to shore, thermally treated to remove base fluid, or land farmed. In some regions, drill cuttings coated with NADF can be discharged into the environment if the base fluid and/or whole mud are approved for discharge. In many cases, cutting dryers are used to remove most of the NADF from the cuttings prior to discharge.

The inability to discharge technically superior NADF and NADF cuttings into the environment presents a huge problem for the oil and gas industry. In many drilling situations, NADFs must be used in order to economically drill the well. This is particularly true with high angle wells, horizontal wells, high pressure high temperature wells, deepwater wells, slimhole wells, and wells drilled into water-sensitive formations.

Many technologies have been developed to deal with the problem of NADF disposal. However, each of these systems has limitations. Cuttings drying is expensive and can only achieve a reduction in oil on cuttings down to 3-4% by weight. The injection of cuttings containing NADF has limitation due to the equipment requirement to capture the cuttings, slurrify them and pump them down an annulus, the lack of available annuli, and the poor understanding of the fracture process involved. Hauling of cuttings containing NADF is expensive and results in non-water quality environmental impacts, including air pollution from transportation, energy use during transportation, and disposal site factors. Landfarming of cuttings requires large areas of land, is a slow process and creates environmental concerns due to the potential for leaching and runoff. Thermal processing of cuttings is expensive, requires a large footprint, and creates safety concerns due to the high temperatures involved. Thus, methodologies that make the drill cuttings more environmentally acceptable would be valuable.

### 3. SUMMARY

The ability of Gordonia sihwensis to sequester and/or biodegrade hydrocarbons is described herein. In an embodiment, provided herein is a biologically pure culture of Gordonia sihwensis. For example, a specific embodiment can be G. sihwensis ATCC PTA-9635. Any technique known to one of skill in the art may be used to obtain a biologically pure culture of bacteria. Generally, a bacterial sample is streaked onto a solid agar-containing medium so as to separate the bacteria present in the sample into individual cells that grow as individual colonies. In one embodiment, a culture of an individual colony from such solid-agar containing medium is considered a biologically pure culture.

One embodiment includes a suitable container or vessel comprising isolated Gordonia sihwensis. In specific embodiments, a container or vessel comprises a biologically pure culture of a Gordonia sihwensis strain, e.g., ATCC PTA-9635. In other embodiments, a container or vessel comprises a mixture of at least one Gordonia sihwensis strain and one or more other microorganisms (e.g., bacterial species). In an embodiment, a container or vessel comprises a mixture of Gordonia sihwensis ATCC PTA-9635 and one or more other microorganisms (e.g., bacterial species). In a specific embodiment, a container or vessel comprises a biologically pure culture of a Gordonia sihwensis strain and a biologically pure culture of one or more other microorganisms (e.g., bacterial species). In a specific embodiment, a container or vessel comprises a biologically pure culture of G. sihwensis ATCC PTA-9635 and a biologically pure culture of one or more other microorganisms (e.g., bacterial species). In certain embodiments, the one or more other microorganisms are capable of sequestering and/or biodegrading hydrocarbons. In certain embodiments, the container or vessel comprises culture medium. In some embodiments, the container or vessel comprises one or more types of hydrocarbons.

In another embodiment, a composition comprises a Gordonia sihwensis strain. In specific embodiments, a composition comprises a biologically pure culture of a G. sihwensis. In an embodiment, a composition comprises a mixture of G. sihwensis strains. In other embodiments, a composition comprises a mixture of a G. sihwensis strain and one or more other microorganisms. In a specific embodiment, the composition comprises a biologically pure culture of a G. sihwensis strain and a biologically pure culture of another microorganism (e.g., bacterial species). In certain embodiments, the other microorganism(s) is capable of sequestering and/or biodegrading hydrocarbons. In certain embodiments, a composition comprises culture medium. In some embodiments, a composition comprises one or more types of hydrocarbons.

In another embodiment, a composition comprises media conditioned by Gordonia sihwensis. In an embodiment, a composition comprises media conditioned by at least one strain of G. sihwensis. In an embodiment, a composition comprises media conditioned by G. sihwensis ATCC PTA-9635. In some embodiments, the conditioned media may be used to sequester hydrocarbons. In one embodiment, a method for sequestering hydrocarbons comprises contacting a hydrocarbon composition with media conditioned by Gordonia sihwensis under conditions which permit the sequestration of the hydrocarbons. In a specific embodiment, the conditioned media is obtained from a culture (e.g., a biologically pure culture) of a Gordonia sihwensis strain while the bacteria are in log phase growth or stationary phase. In an embodiment, a method for sequestering hydrocarbons comprises contacting a hydrocarbon composition with media conditioned by at least G. sihwensis ATCC PTA-9635.

In one aspect, Gordonia sihwensis may be used to sequester hydrocarbons. In one embodiment, in the presence of hydrocarbons, Gordonia sihwensis forms a sac-like structure that surrounds the hydrocarbons. In another embodiment, hydrocarbons are incorporated into a sac-like structure produced by Gordonia sihwensis. In another embodiment, Gordonia sihwensis forms a sac-like structure around hydrocarbons and/or incorporates hydrocarbons into a sac-like structure. In one embodiment, a method for sequestering hydrocarbons comprises contacting a hydrocarbon composition with Gordonia sihwensis under conditions that permit sequestration of hydrocarbons. In another embodiment, a method for sequestering hydrocarbons comprises contacting a hydrocarbon composition with a composition comprising Gordonia sihwensis under conditions which permit sequestration of hydrocarbons. In a specific embodiment, a composition comprising G. sihwensis is a biologically pure culture of Gordonia sihwensis, e.g., G. sihwensis ATCC PTA-9635. In another aspect, Gordonia sihwensis may be used to biodegrade hydrocarbons. Gordonia sihwensis may completely biodegrade hydrocarbons to carbon dioxide or alter the structure of hydrocarbons to produce an intermediate metabolite or biochemical compound. In one embodiment, Gordonia sihwensis transforms an original hydrocarbon structure to carbon dioxide. In another embodiment, Gordonia sihwensis alters an original hydrocarbon structure to form an intermediate metabolite or biochemical compound, such as, e.g., a fatty acid or alcohol. In a specific embodiment, a method for biodegrading hydrocarbons comprises contacting a hydrocarbon composition with Gordonia sihwensis under conditions which permit biodegradation of hydrocarbons. In another embodiment, a method for biodegrading hydrocarbons comprises contacting a hydrocarbon composition with a composition comprising a biologically pure culture of a Gordonia sihwensis strain under conditions which permit the biodegradation of the hydrocarbons. In a specific embodiment, the composition comprises a biologically pure culture of Gordonia sihwensis ATCC PTA-9635.

In another aspect, Gordonia sihwensis is used to sequester and biodegrade hydrocarbons. In a specific embodiment, a method for sequestering and biodegrading hydrocarbons comprises contacting a hydrocarbon composition with Gordonia sihwensis under conditions which permit the sequestration and biodegradation of hydrocarbons. In another embodiment, a method for sequestering and biodegrading hydrocarbons comprises contacting a hydrocarbon composition with a composition comprising Gordonia sihwensis strain ATCC PTA-9635 under conditions which permit the biodegradation of the hydrocarbons. In a specific embodiment, the second composition is a biologically pure culture of Gordonia sihwensis. Non-limiting examples of conditions which permit either the sequestration or biodegradation of hydrocarbons, or both are described herein.

In a specific aspect, the Gordonia sihwensis strain described herein may be used in remediation of drill cuttings coated with drilling fluid. In another aspect, Gordonia sihwensis may be used in the remediation of soil or sludges contaminated with diesel, gasoline, crude oil, or other oil contaminants. In another aspect, Gordonia sihwensis may be used in the clean-up of oil, gasoline or diesel spills. In yet another aspect, Gordonia sihwensis may be used to remove oil, gasoline or diesel from produced water or any quantity water that has been contaminated with oil, gasoline or diesel.

### 4. BRIEF DESCRIPTION OF THE FIGURES

FIG. 1. Ribotype pattern for Gordonia sihwensis strain ATCC PTA-9635.

FIG. 2. A growth curve for Gordonia sihwensis strain ATCC PTA-9635 grown in tryptic soy broth fermentation media.

FIGS. 3A-3G. Microscope photos of aliquots of bacteria taken at 40× magnification at approximately 0 minutes (FIG. 3A), 2 minutes (FIG. 3B), 5 minutes (FIG. 3C), 13 minutes (FIG. 3D), 30 minutes (FIG. 3E), 1 hour (FIG. 3F) and 2 hours (FIG. 3G) after the addition of 2% Estegreen and oil soluble dye to flasks containing Gordonia sihwensis strain ATCC PTA-9635 grown in TSB.

FIGS. 4A-4E. Microscope photos of aliquots of bacteria taken at 40× magnification from flasks with or without different types of surfactant. FIG. 4A. Surfactant-free after 15 minutes; FIG. 4B. 0.02% Triton X-100 after 15 minutes; FIG. 4C. 0.02% Triton X-100 after 2 hours; FIG. 4D. 0.12% Centrolex lecithin after 15 minutes; and FIG. 4E. 0.6% rhamnolipid biosurfactant after 15 minutes.

FIGS. 5A-5C. Microscope photos of aliquots of bacteria taken at 40× magnification from flasks containing 2% Estegreen oil with or without different amounts of drill solids. FIG. 5A. No drill solids; FIG. 5B. 5 grams of drill solids; and FIG. 5C. 10 grams of drill solids.

FIGS. 6A-6F. Microscope photos of aliquots of bacteria taken at 40× magnification from flasks incubated for 15 minutes with or without different types of oil. FIG. 6A. Estegreen; FIG. 6B. Diesel oil; FIG. 6C. Puredrill IA35LV; FIG. 6D. Ametek white oil; FIG. 6E. Kerosene; and FIG. 6F. HDF-2000.

FIGS. 7A-7F. Microscope photos of aliquots of bacteria taken at 40× magnification from flasks incubated for 1 hour with or without different types of oil. FIG. 7A. Estegreen; FIG. 7B. Diesel oil; FIG. 7C. Puredrill IA35LV; FIG. 7D. Ametek white oil; FIG. 7E. Kerosene; and FIG. 7F. HDF-2000.

FIG. 8. Schematic of bioreactor and slurrification tank.

FIG. 9. Percentage of original total petroleum hydrocarbons in the bioreactor.

### 5. DETAILED DESCRIPTION

Described herein is a gram-positive, rod-shaped microorganism, Gordonia sihwensis. A particular strain of G. sihwensis was deposited with the American Type Culture Collection (ATCC), located at 10801 University Boulevard, Manassas, Va. 20110-2209 on Nov. 21, 2008 under the name “Chevron DVAD01”, and assigned ATCC Accession No. PTA-9635.

### 5.1 Culture Conditions for Proliferation of the Bacteria

Gordonia sihwensis may be grown under aerobic conditions. Gordonia sihwensis may be grown in a vessel or container commonly used to culture microorganisms, such as flasks, plates, bioreactors, including by way of example and not limitation, stirred-tank or airlift bioreactors (suspension reactors). In certain embodiments, Gordonia sihwensis strain can be grown in a 5 mL, 10 mL, 20 mL, 50 mL, 100 mL, 200 mL, 500 mL, 1 L, 2 L, 3 L, 4 L, 5 L, 10 L, 100 L, 500 L, 1000 L, 5000 L, 10000 L or 15000 L vessel or container commonly used to culture microorganisms. Gordonia sihwensis may be grown in any vessel or container suitable for laboratory use or commercial use of the bacteria. In a specific embodiment, a biologically pure culture of Gordonia sihwensis can be grown in any vessel or container suitable for laboratory use or commercial use of the bacteria.

Any device used in the art for maintaining culture conditions (such as temperature, pH, oxygenation, etc.) may be used as part of, or in conjunction with, a vessel or container commonly used to culture microorganisms. In a specific embodiment, the temperature of a G. sihwensis culture can be maintained at approximately 25° C. to approximately 45° C., approximately 30° C. to approximately 45° C., approximately 35° C. to approximately 45° C., approximately 35° C. to approximately 40° C. In another embodiment, the temperature of the culture is maintained at approximately 30° C., 31° C., 32° C., 33° C., 34° C., 35° C., 36° C., 37° C., 38° C., 39° C., 40° C., 41° C., 42° C., 43° C., 44° C. or 45° C. In certain embodiments, the pH of the culture medium is monitored during the culture process so that the pH remains at approximately pH 6.0 to approximately pH 8.0, approximately pH 6.8 to approximately pH 7.6, approximately pH 7.0 to approximately pH 7.6, approximately pH 7.0 to approximately pH 7.4, approximately pH 7.0 to approximately pH 7.2 or approximately pH 7.0. In another embodiment, the bacterial culture is shaken at approximately 10 rpm to approximately 25 rpm, approximately 25 to approximately 50 rpm, approximately 25 to approximately 75 rpm, approximately 50 to approximately 100 rpm, or approximately 75 rpm to approximately 100 rpm. In other embodiments, the bacterial culture is shaken at approximately 100 rpm to approximately 400 rpm or approximately 150 rpm to approximately 300 rpm in the vessel or container. Sufficient aeration is provided to the bacterial culture to maintain a sufficient concentration of dissolved oxygen. In a specific embodiment, sufficient aeration is provided to maintain a dissolved oxygen concentration of approximately 0.5 mg/L to approximately 25 mg/L, approximately 1 mg/L to approximately 25 mg/L, approximately 1 mg/L to approximately 20 mg/L, approximately 1 mg/L to approximately 15 mg/L, approximately 1 mg/L to approximately 10 mg/L, approximately 1 mg/L to approximately 5 mg/L, or approximately 5 mg/L to approximately 20 mg/L.

As used herein, the terms “about” and “approximately”, unless otherwise indicated, refer to a value that is no more than 20% above or below the value being modified by the term.

Any microbial culture medium known in the art may be suitable to grow Gordonia sihwensis. The suitability of a particular microbial culture medium can be determined using methods known in the art or described herein. For example, the suitability of a particular medium may be determined by assessing the proliferation of the bacteria or the ability of the bacteria to form sac-like structures. In one embodiment, the culture media is nutrient broth. In another embodiment, the culture media is tryptic soy broth (TSB). In another embodiment, the culture media is 50/50 TSB/enhanced Inakollu mineral media. In another embodiment, the culture media is brain heart infusion (BHI) broth. In certain embodiments, Gordonia sihwensis can be grown in medium in which the sole carbon source is a hydrocarbon. In some embodiments, Gordonia sihwensis can be grown in medium in which the sole carbon source is a mixture of two or more types of hydrocarbons.

Any technique known in the art may be used to inoculate a suitable microbial culture medium. The amount bacteria in an inoculum can vary depending upon a number of factors, including, e.g., the size of the vessel or container and the volume of the culture medium. In a specific embodiment, an inoculum of approximately 5,000 colony forming units (CFU) to approximately 50,000,000 CFU, approximately 5,000 CFU to approximately 40,000,000 CFU, approximately 5,000 CFU to approximately 30,000,000 CFU, approximately 5,000 CFU to approximately 25,000,000 CFU, approximately 5,000 CFU to approximately 15,000,000 CFU or approximately 5,000 CFU to approximately 10,000,000 CFU is used to inoculate a suitable microbial cell culture medium. In another embodiment, an inoculum of approximately 10,000 CFU to approximately 50,000,000 CFU, approximately 10,000 CFU to approximately 40,000,000 CFU, approximately 10,000 CFU to approximately 30,000,000 CFU, approximately 10,000 CFU to approximately 25,000,000 CFU, approximately 10,000 CFU to approximately 15,000,000 CFU or approximately 10,000 CFU to approximately 10,000,000 CFU is used to inoculate a suitable microbial cell culture medium. In another embodiment, an inoculum of approximately 25,000 CFU to approximately 50,000,000 CFU, approximately 25,000 CFU to approximately 40,000,000 CFU, approximately 25,000 CFU to approximately 30,000,000 CFU, approximately 25,000 CFU to approximately 25,000,000 CFU, approximately 25,000 CFU to approximately 15,000,000 CFU or approximately 25,000 CFU to approximately 10,000,000 CFU is used to inoculate a suitable microbial cell culture medium. In yet another embodiment, an inoculum of approximately 10,000 CFU to approximately 5,000,000 CFU, approximately 10,000 CFU to approximately 2,000,000 CFU, approximately 10,000 CFU to approximately 1,000,000 CFU, approximately 10,000 CFU to approximately 750,000 CFU, approximately 10,000 CFU to approximately 500,000 CFU or approximately 10,000 CFU to approximately 250,000 CFU is used to inoculate a suitable microbial cell culture medium.

### 5.2 Sequestration and Biodegradation

In one aspect, a composition comprising media conditioned by Gordonia sihwensis may be used to sequester hydrocarbons. In one embodiment, a method for sequestering hydrocarbons comprises contacting a hydrocarbon composition with media conditioned by Gordonia sihwensis under conditions which permit sequestration of the hydrocarbons. In a specific embodiment, the conditioned media is obtained from a biologically pure culture of a Gordonia sihwensis strain (e.g., strain ATCC PTA-9635). The conditioned media can be from when G. sihwensis is in log phase or stationary phase.

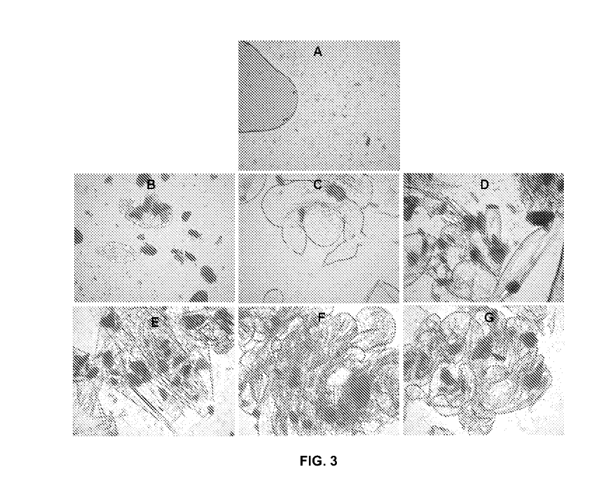

In another aspect, Gordonia sihwensis is capable of sequestering hydrocarbons. In one embodiment, Gordonia sihwensis forms a sac-like structure around hydrocarbons, such as the sac-like structures shown in FIGS. 3-7. In another embodiment, hydrocarbons are incorporated into a sac-like structure produced by Gordonia sihwensis. In a specific embodiment, in microbial culture medium, Gordonia sihwensis forms a sac-like structure around hydrocarbons and/or incorporates hydrocarbons into a sac-like structure.

In one embodiment, a method for sequestering hydrocarbons comprises contacting a hydrocarbon composition with a culture or an inoculum of the Gordonia sihwensis strain described herein under conditions which permit the sequestration of the hydrocarbon(s) present in the composition. In another embodiment, a method for sequestering hydrocarbons comprises contacting a hydrocarbon composition with a composition comprising Gordonia sihwensis under conditions which permit sequestration of hydrocarbon(s) present in the composition. In a specific embodiment, a bacterial composition is a biologically pure culture of a Gordonia sihwensis strain, for example, ATCC PTA-9635. Non-limiting examples of conditions which permit the sequestration of a hydrocarbon(s) are described below.

In one embodiment, the capability of Gordonia sihwensis or medium conditioned by the bacteria to sequester hydrocarbons is assessed by a technique known to one of skill in the art. In some embodiments, the technique used is one that is used to assess the presence of a biosurfactant. In a specific embodiment, the capability of the Gordonia sihwensis strain described herein or medium conditioned by the bacteria to sequester hydrocarbons is assessed using one of the assays described in the example section herein.

Without being bound by any theory, sequestration of hydrocarbons by Gordonia sihwensis into sac-like structures may be advantageous because: (1) inefficient contact between bacteria and hydrocarbons has been a long-standing limitation in hydrocarbon biodegradation, and (2) the sac-like structures may remove hydrocarbons from the environment for a period of time.

In certain embodiments, sequestration of hydrocarbons by Gordonia sihwensis can be in rich media (i.e., media that contains a carbon source other than the hydrocarbons being sequestered or biodegraded such as meat extract or peptide extract). Without being bound by any theory, sequestration of hydrocarbons by Gordonia sihwensis in rich media may be advantageous because the bacteria can be grown to a high population density in a relatively short period of time. In some embodiments, sequestration of hydrocarbons by Gordonia sihwensis is in lean media (e g., mineral media such as Inakollu media or enhanced Inakollu media). Without being bound by any theory, sequestration of hydrocarbons by Gordonia sihwensis in lean media (e g., mineral media such as Inakollu media or enhanced Inakollu media) may not be as advantageous as rich media because there may be a longer lag period for growth in lean media.

In another aspect, Gordonia sihwensis biodegrades hydrocarbons. In a specific embodiment, in microbial culture medium, Gordonia sihwensis biodegrades hydrocarbons when hydrocarbons are added to the media. Gordonia sihwensis may completely biodegrade hydrocarbons to carbon dioxide or alter the structure of hydrocarbons to produce an intermediate metabolite or biochemical compound. In one embodiment, Gordonia sihwensis transforms an original hydrocarbon structure to carbon dioxide. In another embodiment, Gordonia sihwensis alters an original hydrocarbon structure to form an intermediate metabolite or biochemical compound, such as, e.g., a fatty acid or alcohol.

In certain embodiments, biodegradation of hydrocarbons by Gordonia sihwensis occurs in rich media (i.e., media that contains a carbon source other than the hydrocarbons being sequestered or biodegraded such as meat extract or peptide extract). In some embodiments, biodegradation of hydrocarbons by the Gordonia sihwensis occurs in lean media (e.g., mineral media such as Inakollu media or enhanced Inakollu media).

In a specific embodiment, a method for biodegrading hydrocarbons comprises contacting a hydrocarbon composition with a culture or an inoculum of Gordonia sihwensis under conditions which permit biodegradation of hydrocarbon(s) present in a composition. In another embodiment, a method for biodegrading hydrocarbons comprises contacting a hydrocarbon composition with a composition comprising Gordonia sihwensis under conditions which permit the sequestration of the hydrocarbon(s) present in the composition. In a specific embodiment, the bacterial composition is a biologically pure culture of a Gordonia sihwensis strain, for example strain ATCC PTA-9635. Non-limiting examples of conditions which permit biodegradation of a hydrocarbon(s) are described below.

In one embodiment, the capability of Gordonia sihwensis to biodegrade hydrocarbons is assessed by a technique known to one of skill in the art. In a specific embodiment, the capability of Gordonia sihwensis to biodegrade hydrocarbons is assessed using a total petroleum hydrocarbon (TPH) assay, such as the TPH assay referenced in the example section herein. The TPH assay referenced in the example below provides the percentage of total hydrocarbons recovered; the percentage of hydrocarbons biodegraded may be obtained by subtracting the percentage of total hydrocarbons recovered from 100%.

In another aspect, Gordonia sihwensis sequesters hydrocarbons and biodegrades hydrocarbons. In one embodiment, a method for sequestering and biodegrading hydrocarbons comprises contacting a hydrocarbon composition with a culture or an inoculum of Gordonia sihwensis under conditions which permit the sequestration and biodegradation of the hydrocarbon(s) present in the composition. In another embodiment, a method for sequestering and biodegrading hydrocarbons comprises contacting a hydrocarbon composition with a composition of Gordonia sihwensis under conditions which permit sequestration and biodegradation of hydrocarbon(s) present in the composition. In a specific embodiment, the bacterial composition is a biologically pure culture of a Gordonia sihwensis strain, e.g., ATCC PTA-9635. Non-limiting examples of conditions which permit the sequestration and biodegradation of a hydrocarbon(s) are described below.

In certain embodiments, an inoculum of Gordonia sihwensis is contacted with a hydrocarbon composition in a vessel, tank or other suitable container (e.g., a bioreactor). In other embodiments, a hydrocarbon composition is contacted with a composition comprising a culture of Gordonia sihwensis in a vessel, tank (e.g., slurrification tank) or other suitable container (e.g., a bioreactor or flask) after the bacteria have been permitted to proliferate. In a specific embodiment, a hydrocarbon composition is contacted with a composition comprising a biologically pure culture of Gordonia sihwensis in a vessel, tank (e.g., slurrification tank) or other suitable container (e.g., a bioreactor or flask). In another embodiment, a hydrocarbon composition is contacted with a composition comprising Gordonia sihwensis and one or more other microorganisms (e.g., bacterial species) in a vessel, tank (e.g., slurrification tank) or other suitable container (e.g., a bioreactor or flask). In certain embodiments, the one or more other microorganisms are capable of sequestering and/or biodegrading oil. In an embodiment, the biologically pure culture is a culture of G. sihwensis ATCC PTA-9635.

In certain embodiments, a hydrocarbon composition is contacted with a composition comprising Gordonia sihwensis in a vessel, tank (e.g., slurrification tank) or other suitable container (e.g., bioreactor or flask) after the bacteria have entered log phase in their growth (e.g., approximately 6 hours to approximately 18 hours, approximately 8 hours to approximately 16 hours, approximately 10 hours to approximately 18 hours, or approximately 12 hours to approximately 18 hours after inoculating the bacteria into the culture medium). In specific embodiments, a hydrocarbon composition is contacted with a composition comprising a biologically pure culture of a Gordonia sihwensis strain in log phase growth in a vessel, tank (e.g., slurrification tank) or other suitable container (e.g., bioreactor or flask). In some embodiments, a hydrocarbon composition is contacted with a composition comprising a Gordonia sihwensis strain in log phase growth and one or more other microorganisms (e.g., bacterial species) in a vessel, tank (e.g., slurrification tank) or other suitable container (e.g., bioreactor or flask). In certain embodiments, the one or more other microorganisms are capable of sequestering and/or biodegrading oil. In an embodiment, the G. sihwensis strain is ATCC PTA-9635.

In certain embodiments, a hydrocarbon composition is contacted with a composition comprising Gordonia sihwensis in a vessel, tank (e.g., slurrification tank) or other suitable container (e.g., a bioreactor or flask) after the bacteria have entered the stationary phase in their growth (e.g., approximately 18 hours to approximately 22 hours or approximately 18 hours to approximately 24 hours after inoculating the bacteria into the culture medium). In specific embodiments, a hydrocarbon composition is contacted with a composition comprising a biologically pure culture of Gordonia sihwensis in the stationary phase of growth in a vessel, tank (e.g., slurrification tank) or other suitable container (e.g., a bioreactor or flask). In some embodiments, a hydrocarbon composition is contacted with a composition comprising Gordonia sihwensis in the stationary phase of growth and one or more other microorganisms (e.g., bacterial species) in a vessel, tank (e.g., slurrification tank) or other suitable container (e.g., a bioreactor or flask). In certain embodiments, the one or more other microorganisms are capable of sequestering and/or biodegrading oil. In an embodiment, the G. sihwensis is G. sihwensis strain ATCC PTA-9635.

In some embodiments, a hydrocarbon composition is contacted with a composition comprising a suitable microbial culture medium and Gordonia sihwensis in a vessel, tank (e.g., slurrification tank) or other suitable container (e.g., a bioreactor or flask) after the bacteria have been permitted to proliferate. In a specific embodiment, a hydrocarbon composition is contacted with a composition comprising a suitable microbial culture medium and Gordonia sihwensis in a vessel, tank (e.g., slurrification tank) or other suitable container (e.g., bioreactor or flask) after the bacteria have entered the log phase in their growth (e.g., approximately 6 hours to approximately 18 hours, approximately 8 hours to approximately 16 hours, approximately 10 hours to approximately 18 hours, or approximately 12 hours to approximately 18 hours after inoculating the bacteria into the culture medium). In another specific embodiment, a hydrocarbon composition is contacted with a composition comprising a suitable microbial culture medium and Gordonia sihwensis in a vessel, tank (e.g., slurrification tank) or other suitable container (e.g., a bioreactor or flask) after the bacteria have entered the stationary phase in their growth (e.g., approximately 18 hours to approximately 22 hours or approximately 18 hours to approximately 24 hours after inoculating the bacteria into the culture medium).

The vessel, tank, or container in which a bacterial composition and a hydrocarbon composition are combined can be any vessel, tank or container commonly used to culture microorganisms, such as flasks or bioreactors, including by way of example and not limitation, stirred-tank or airlift bioreactors (suspension reactors). In certain embodiments, the vessel or container is a 5 mL, 10 mL, 20 mL, 50 mL, 100 mL, 200 mL, 500 mL, 1 L, 2 L, 3, L, 4 L, 5L, 10 L, 100 L, 500 L, 1000 L, 5000 L, 10000 L or 15000 L vessel, tank or container commonly used to culture microorganisms. The vessel or container may be suitable for laboratory use or commercial use.

In some embodiments, a hydrocarbon composition and a composition comprising Gordonia sihwensis are mixed in a slurrification tank and then transferred to a bioreactor. In certain embodiments, a hydrocarbon composition and a composition comprising Gordonia sihwensis are mixed in a slurrification tank for approximately 30 minutes to approximately 10 hours, approximately 30 minutes to approximately 5 hours, or approximately 30 minutes to approximately 3 hours and then transferred to a bioreactor.

Any device used in the art for maintaining culture conditions (such as temperature, pH, oxygenation, etc.) may be used as part of, or in conjunction with, a vessel, tank or container commonly used to culture microorganisms. In a specific embodiment, the temperature of the bacterial/hydrocarbon composition mixture is maintained at approximately 25° C. to approximately 45° C., approximately 30° C. to approximately 45° C., approximately 35° C. to approximately 45° C., approximately 35° C. to approximately 40° C. In another embodiment, the temperature of the bacterial/hydrocarbon composition mixture is maintained at approximately 30° C., 31° C., 32° C., 33° C., 34° C., 35° C., 36° C., 37° C., 38° C., 39° C., 40° C., 41° C., 42° C., 43° C., 44° C. or 45° C. In certain embodiments, the pH of the bacterial/hydrocarbon composition mixture is maintained at approximately pH 6.0 to approximately pH 8.0, approximately pH 6.8 to approximately pH 7.6, approximately pH 7.0 to approximately pH 7.6, approximately pH 7.0 to approximately pH 7.4, approximately pH 7.0 to approximately pH 7.2 or approximately pH 7.0. In another embodiment, the bacterial/hydrocarbon composition is shaken at approximately 10 rpm to approximately 25 rpm, approximately 25 to approximately 50 rpm, approximately 25 to approximately 75 rpm, approximately 50 to approximately 100 rpm, or approximately 75 rpm to approximately 100 rpm. In another embodiment, the bacterial/hydrocarbon composition mixture is shaken at approximately 100 rpm to approximately 400 rpm or approximately 150 rpm to approximately 300 rpm in the vessel or container. In a specific embodiment, the bacterial/hydrocarbon composition mixture is shaken at approximately 150 rpm or 300 rpm. In another embodiment, sufficient aeration is provided to maintain a sufficient concentration of dissolved oxygen in the vessel or container. In a specific embodiment, sufficient aeration is provided to maintain a dissolved oxygen concentration of approximately 0.5 mg/L to approximately 25 mg/L, approximately 1 mg/L to approximately 25 mg/L, approximately 1 mg/L to approximately 20 mg/L, approximately 1 mg/L to approximately 15 mg/L, approximately 1 mg/L to approximately 10 mg/L, approximately 1 mg/L to approximately 5 mg/L, or approximately 5 mg/L to approximately 20 mg/L.

### 5.3 Hydrocarbon Compositions

As used herein, the term “hydrocarbon composition” refers to a composition comprising a quantity of at least one hydrocarbon. In a specific embodiment, a hydrocarbon composition comprises one, two, three or more hydrocarbons. In another embodiment, a hydrocarbon composition comprises only one type of hydrocarbon. In another embodiment, a hydrocarbon composition comprises two or more types of hydrocarbons. In another embodiment, a hydrocarbon composition comprises a mixture or combination of different types of hydrocarbons.

In certain embodiments, approximately 0.5% to approximately 65%, approximately 1% to approximately 65%, approximately 5% to approximately 65%, approximately 10% to approximately 65%, approximately 25% to approximately 65% or approximately 30% to approximately 65% of a hydrocarbon composition is composed of one or more hydrocarbons. In some embodiments, approximately 5% to approximately 30%, approximately 10% to approximately 30%, approximately 15% to approximately 30%, approximately 20% to approximately 30%, or approximately 25% to approximately 30% of a hydrocarbon composition is composed of one or more hydrocarbons. In other embodiments, approximately 5% to approximately 30%, approximately 0.5% to approximately 15%, approximately 0.5% to approximately 10%, approximately 0.5% to approximately 5%, or approximately 0.5% to approximately 2% of a hydrocarbon composition is composed of one or more hydrocarbons.

In certain embodiments, a particular hydrocarbon accounts for approximately 0.5% to approximately 95%, approximately 10% to approximately 95%, approximately 25% to approximately 95%, approximately 50% to approximately 95%, or approximately 75% to approximately 95% of the total hydrocarbon content in a hydrocarbon composition. In some embodiments, a particular hydrocarbon accounts for approximately 10% to approximately 75%, approximately 10% to approximately 50%, approximately 10% to approximately 25%, approximately 25% to approximately 50%, or approximately 50% to approximately 75% of the total hydrocarbon content in a hydrocarbon composition.

In certain embodiments, a hydrocarbon composition comprises two or more types of hydrocarbons with each hydrocarbon accounting for a certain percentage of the total hydrocarbon content of the composition. In some embodiments, a first type of hydrocarbon accounts for approximately 0.5% to approximately 15% of the total hydrocarbon content of a hydrocarbon composition and a second type of hydrocarbon accounts for approximately 85% to approximately 95% of the total hydrocarbon content in a hydrocarbon composition. In other embodiments, a first type of hydrocarbon accounts for approximately 10% to approximately 40% of the total hydrocarbon content of a hydrocarbon composition and a second type of hydrocarbon accounts for approximately 60% to 90% of the total hydrocarbon content of a hydrocarbon composition. In other embodiments, a first type of hydrocarbon accounts for approximately 25% to approximately 60% of the total hydrocarbon content of a hydrocarbon composition and a second type of hydrocarbon accounts for approximately 40% to approximately 75% of the total hydrocarbon content of a hydrocarbon composition. Hydrocarbons include, but are not limited to, aliphatic hydrocarbons, aromatic hydrocarbons, nitro-aromatic hydrocarbons, halo-aliphatic hydrocarbons and halo-aromatic hydrocarbons. Non-limiting examples of hydrocarbons include alkenes (e.g., methane, ethane, propane, butane, isobutane, pentane, isopentane, neopentane, hexane, octane, nonane, and decane), alkenes (e.g., ethene, propene, butene, pentene, hexane, heptene, octane, nonene, and decene), alkynes (e.g., ethyne, propyne, butyne, pentyne, hexyne, heptyne, octyne, nonyne, and decyne), cycloalkanes (e.g., cyclopropane, cyclobutane, methylcyclopropane, cyclopentane, cyclohexane, cycloheptane, methylcyclohexane, cyclooctane, cyclononane and cyclodecane), alkadienes (e.g., allene, butadiene, pentadiene, isoprene, hexadiene, heptadiene, octadiene, nonadiene, and decadiene), and aromatic hydrocarbons (e.g., benzene, naphthalene, anthracene, toluene, xylenes, ethylbenzene, methylnaphthalene, aniline, phenol, and dimethylphenol).

In a specific embodiment, a hydrocarbon composition comprises one, two or more, or a combination of a C10 to C20 n-alkane, a C10 to C20 n-alkene, and an isoalkane. In another embodiment, a hydrocarbon composition comprises one, two or more of the following: decane, undecane, dodecane, tridecane, tetradecane, pentadecane, and hexadecane, heptadecane, octadecane, nonadecane and eicosane. In another embodiment, a hydrocarbon composition comprises one, two or more of the following: isobutene, 2,4-dimethylpentane, isooctane, and 2,2,4-trimethyldecane. In another embodiment, a hydrocarbon composition comprises a paraffin. In another embodiment, a hydrocarbon composition comprises an isoparaffin. In another embodiment, a hydrocarbon composition comprises a cycloparaffin. In certain embodiments, a hydrocarbon composition comprises a mixture of isoparaffins, n-paraffins, and cycloparaffins. In some embodiments, a hydrocarbon composition comprises a mixture of isoparaffins, n-paraffins, cycloparaffins and aromatics.

In a specific embodiment, a hydrocarbon composition comprises a base oil. Base oils include, but are not limited to, synthetic base oils, mineral base oils and diesel. Non-limiting examples of synthetic base oils include Estegreen (Chevron), Ecoflow (Chevron), Saraline (Shell MDS), Mosspar H (PetroSA), Sarapar (Shell MDS), Baroid Alkane (Halliburton), XP-07 (Halliburton), Inteq (Baker Hughes Drilling Fluids), Novadrill (M-I Swaco), Biobase (Shrieve Chemicals), Sasol C1316 paraffin (Sasol), Isoteq (Baker Hughes Drilling Fluids), Amodrill (BP Chemicals), Petrofree Ester (Halliburton), Finagreen Ester (Fina Oil and Chemical), CPChem internal olefins (ChevronPhillips Chemical), and Neoflo olefins (Shell Chemicals). Non-limiting examples of mineral oils include Escaid (Exxon), Vassa LP (Vassa), EDC-95-11 (Total), EDC99-DW (Total), HDF-2000 (Total), Mentor (Exxon), LVT (ConocoPhillips), HDF (Total), BP 83HF (BP), DMF 120HF (Fina), DF-1 (Total), EMO 4000, Shellsol DMA (Shell), IPAR 35 LV (PetroCanada), IPAR 35 (PetroCanada), Telura 401 (Exxon), SIPDRILL (SIP Ltd.), Puredrill® IA35LV, white oil (Ametek; Paoli, PA), and Clairsol (Carless Solvents). Other examples of base oils include, but are not limited to, crude oil, diesel oil, Ametek® (Ametek; Paoli, PA), Isomerized Alpha Olefin C₁₆ (Chevron Phillips Chemical Company), Isomerized Alpha Olefin C₁₈ (Chevron Phillips Chemical Company), Isomerized Alpha Olefin C₁₆₋₁₈(_{65:35}) (Chevron Phillips Chemical Company), and kerosene.

In some embodiments, a hydrocarbon composition does not contain a surfactant. In other embodiments, a hydrocarbon composition comprises a surfactant. As used herein, the term “surfactant” refers to organic substances having amphipathic structures (namely, they are composed of groups of opposing solubility tendencies, typically an oil-soluble hydrocarbon chain and a water-soluble ionic group) which have the property of adsorbing onto the surfaces or interfaces of a system and of altering to a marked degree the surface or interfacial free energies of those surfaces (or interfaces) As used in the foregoing definition of surfactant, the term “interface” indicates a boundary between any two immiscible phases and the term “surface” denotes an interface where one phase is a gas, usually air. Surfactants can be classified, depending on the charge of the surface-active moiety, into anionic, cationic, and nonionic surfactants. Surfactants are often used as wetting, emulsifying, solubilizing, and dispersing agents. Non-limiting examples of surfactants include fatty acids, soaps of fatty acids, fatty acid derivates, lecithin, crude tall oil, oxidized crude tall oil, organic phosphate esters, modified imidazolines, modified amidoamines, alkyl aromatic sulfates, alkyl aromatic sulfonates, organic esters, and polyhydric alcohols. Other examples of surfactants include amido-amines, polyamides, polyamines, esters (such as sorbitan, monooleate polyethoxylate, and sorbitan dioleate polyethoxylate), imidazolines, and alcohols.

In certain embodiments, a hydrocarbon composition does not contain CaCl₂. In other embodiments, a hydrocarbon composition comprises CaCl₂.

In a specific embodiment, a hydrocarbon composition comprises a drilling fluid. In one embodiment, the drilling fluid is a water based drilling fluid. In another embodiment, the drilling fluid is a non-aqueous drilling fluid. Non-limiting examples of drilling fluids include Aqua-Drill™ (Baker Hughes), Plus System (Baker Hughes), Aqua-Drill™ System (Baker Hughes), Bio-Lose 90 System v, Carbo-Core System(Baker Hughes), Carbo-Drill® System (Baker Hughes), Clear-Drill® DIF System (Baker Hughes), Deep Water Fluid System (Baker Hughes), Max-Bridge^{SM} System (Baker Hughes), Micro-Prime^{SM} System (Baker Hughes), New-Drill® System (Baker Hughes), OMNIFLOW® DIF System (Baker Hughes), PERFFLOW® 100 DIF System (Baker Hughes), PERFFLOW® DIF System (Baker Hughes), PERFFLOW® HD DIF System (Baker Hughes), PERFFLOW® System (Baker Hughes), PERFORMAX^{SM} System (Baker Hughes), PYRO-Drill® System (Baker Hughes), RHEO-Logic^{SM} System (Baker Hughes), SCIFLOW™ DIF System (Baker Hughes), SYN-TEQ® System (Baker Hughes), and TERRA-MAX^{SM} System (Baker Hughes). In a specific embodiment, the drilling fluid is a synthetic or mineral-based drilling fluid. Examples of synthetic and mineral-based drilling fluids include, but are not limited to, Petrofree (Halliburton), Petrofree LV (Halliburton), Petrofree SF (Halliburton), Coredril-N (Halliburton), Encore (Halliburton), Integrade (Halliburton), Innovert (Halliburton), Accolade (Halliburton), Versadril (M-I Swaco), Versaclean (M-I Swaco), Paraland (M-I Swaco), Ecogreen (M-I Swaco), Trudrill (M-I Swaco), Novapro (M-I Swaco), Novatec (M-I Swaco), Trucore (M-I Swaco), Parapro (M-I Swaco), Versapro (M-I Swaco), Versapro LS (M-I Swaco), Rheliant (M-I Swaco), Magma-Drill (Baker Hughes), Magma-Teq (Baker Hughes), Syn-Core (Baker Hughes), Optidrill (Newpark), Optiphase (Newpark), Cyberdrill (Newpark), Cyberphase (Newpark), Confi-Drill (SCOMI), Confi-Dense (SCOMI), Extra-Vert (SCOMI), Opta-Vert (SCOMI), and Opta-Vert 100 (SCOMI).

In a specific embodiment, a hydrocarbon composition comprises drill cuttings. In another specific embodiment, a hydrocarbon composition comprises or is a petroleum product, such as oil, gasoline or diesel. In another embodiment, a hydrocarbon composition comprises water contaminated with one or more hydrocarbons, such as oil, gasoline or diesel. In another embodiment, a hydrocarbon composition comprises soil or sludge contaminated with one or more hydrocarbons.

### 5.4 Storage of Bacteria

Gordonia sihwensis may be stored under any conditions that preserve the viability of the strain. Techniques for storing bacteria are well-known to one of skill in the art. In one embodiment, Gordonia sihwensis is frozen in Brucella/glycerol and stored at approximately −70° C. to approximately -80° C. or in a liquid nitrogen tank. Frozen cultures of G. sihwensis may be thawed, streaked onto a trypticase soy agar (TSA) or a TSA sheep's blood agar plate, or a TSA/Estegreen base oil agar plate and incubated at about 35° C. prior to use. In a specific embodiment, the G. sihwensis is sub-cultured every 3 to 10 days to prevent overgrowth on the agar plates.

### 5.5 Kits

In one aspect, described herein is a kit comprising, in a container (e.g., a vial or plate), Gordonia sihwensis. In an embodiment, the G. sihwensis is a biologically pure culture. In an embodiment, the G. sihwensis is G. sihwensis ATCC PTA-9635. In a specific embodiment, described herein is a kit comprising, in a container (e.g., a vial or plate), a biologically pure culture of Gordonia sihwensis. In another embodiment, provided herein is a kit comprising, in one or more containers, Gordonia sihwensis and one or more other microorganisms (e.g., one or more bacterial species). In certain embodiments, the one or more other microorganisms are capable of sequestering and/or biodegrading oil. In specific embodiments, the kit further comprises instructions for use of Gordonia sihwensis. For example, in certain embodiments, the kit includes instructions for growing the bacteria, sequestering hydrocarbons and/or biodegrading hydrocarbons.

### EXAMPLES

6.1 Gordonia sihwensis Strain

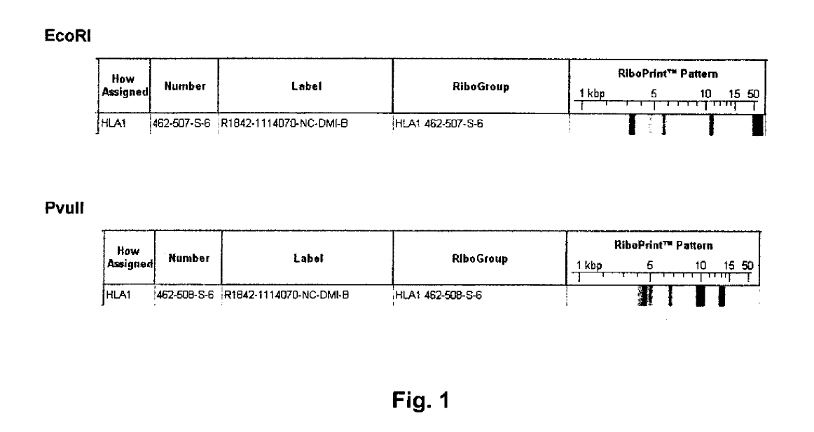

Gordonia sihwensis strain ATCC PTA-9635 (also known as G. sihwensis Chevron DVAD01) was isolated from a biopile in Texas. The bacterial strain is a gram-positive, rod-shaped microorganism from the species Gordonia sihwensis. The ribotyping results for the deposited strain are shown in FIG. 1.

### 6.2 Growth Characteristics of the Bacteria

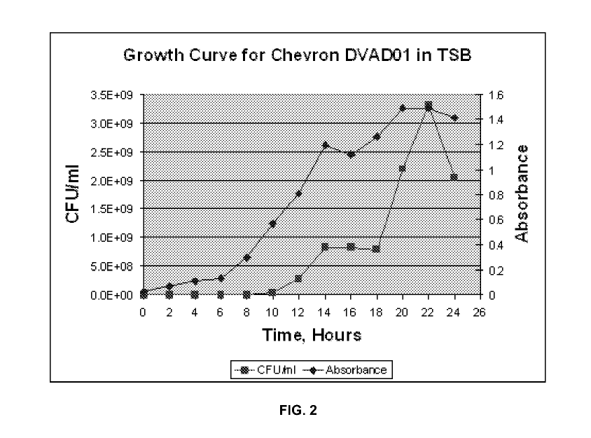

Approximately two loopfuls of Gordonia sihwensis strain ATCC PTA-9635 (which is approximately 20,000 CFU to approximately 5,000,000 CFU) were inoculated into flasks containing 50 mL of 100% tryptic soy broth (TSB) fermentation media (EMD Chemicals; Gibbstown, N.J.) The flasks were shaken at 35° C. at 150 rpm. Aliquots of 1 mL were taken at approximately 0, 2, 4, 6, 8, 10, 12, 14, 16, 18, 20 22, and 24 hours after inoculation and spectrophotometry readings at 590 nm were performed. In addition, aliquots of 1 mL were taken at each time point (i.e., 0, 2, 4, 6, 8, 10, 12, 14 16, 18, 20, 22 and 24 hours after inoculation of the TSB), diluted in sterilized deionized water to various concentrations and plated onto tryptic soy agar (TSA). The TSA plates were incubated at 35° C. for 24 hours and colony forming units (CFU) were counted. As shown in FIG. 2, the bacteria entered log phase between about 8 and 14 hours and after a brief stationary phase between about 14 and 18 hours, entered a second log phase between about 18 and 22 hours. At about 22 hours, the CFU decreased indicating that the viability of the bacteria had decreased.

### 6.2 Sac-Like Structure Formation

G. sihwensis strain ATCC PTA-9635 forms a sac-like structure in growth media when base oil is added to the media-bacteria. Approximately two loopfuls of G. sihwensis strain ATCC PTA-9635 (which is approximately 20,000 CFU to approximately 5,000,000 CFU) was inoculated into a flask containing 50 mL of TSB and the flask was incubated at 35° C. at 150 rpm. After approximately 22 hours, 1 mL of base oil (2 vol. % of Estegreen (Chevron) in microbial culture medium) and 0.02 mL of Oil Red 0 (EMD Chemicals; Gibbstown, N.J.), an oil soluble dye, were added to the flask and the flask was shaken at 35° C. at 150 rpm. The oil soluble dye was added to the flask to visually observe the base oil added to the flask. The dye is red in the presence of oil. Approximately 0 minutes, 2 minutes, 5 minutes, 13 minutes, 30 minutes, 1 hour and 2 hours after the addition of the base oil and oil soluble dye, aliquots of the bacteria were taken from the flask and photos of the bacteria at 40× magnitude under the microscope were taken (see FIGS. 3A-3G). At approximately 0 minutes, free and clumped bacteria are observed, and the oil soluble dye is clearly visible. As shown in FIG. 3B, approximately 2 minutes after the addition of the base oil and oil soluble dye to the flask, sac-like structures begin to form and oil soluble dye becomes less visible. As time lapses, the sac-like structures become more structured and the oil soluble dye becomes less visible. By approximately 5 minutes after the addition of the base oil and oil soluble dye to the flask, the sac-like structures are well formed (FIG. 3C). Approximately 30 minutes after the addition of the base oil and oil soluble dye, an extensive network of stretched and collapsed sac-like structures are observed (FIG. 3E). Without being bound by any theory, it is believed that the sac-like structures form when the base oil is added to the flask to gather and trap the oil.

### 6.3 Effect of Growth Conditions on Formation of Sac-Like Structures

### Effect of Shaking on Formation of Sac-Like Structures

The effect of shaking at 150 rpm after the addition of base oil (2 vol. % base oil in microbial culture medium) and shaking at 300 rpm after the addition of base oil (2 vol. % of Estegreen (Chevron) in microbial culture medium) on the formation of sac-like structures was compared. Approximately two loopfuls of G. sihwensis strain ATCC PTA-9635 described herein (which is approximately 20,000 CFU to approximately 5,000,000 CFU) was inoculated into two flasks, each flask containing 50 mL of 100% TSB, and each flask was incubated at 35° C. at 150 rpm. After approximately 20 hours, 1 mL of base oil (2 vol. % of Estegreen (Chevron) in microbial culture medium) and 0.02 mL of Oil Red 0 (EMD Chemicals; Gibbstown, N.J.) were added to each flask. One flask was shaken at 35° C. at 150 rpm and the other flask was shaken at 35° C. at 300 rpm. After certain periods of time, aliquots were taken from each flask and the formation of the sac-like structures and visibility of the oil soluble dye was observed using a microscope. Although the sac-like structures formed more quickly in the flask shaken at 300 rpm, there was no noticeable difference between the flask shaken at 150 rpm and the flask shaken at 300 rpm after 10 minutes. The effect of shaking at 150 rpm while growing the bacteria overnight before the addition of base oil (2 vol. % of Estegreen (Chevron) in microbial culture medium) was compared to the effect of shaking at 300 rpm while growing the bacteria overnight before the addition of base oil (2 vol. % of Estegreen (Chevron) in microbial culture medium) on the formation of sac-like structures was compared. Approximately two loopfuls of G. sihwensis strain ATCC PTA-9635 (which is approximately 20,000 CFU to approximately 5,000,000 CFU) were inoculated into two flasks, each flask containing 50 mL of 100% TSB. One flask was shaken at 35° C. at 150 rpm and the other flask was shaken at 35° C. at 300 rpm. After approximately 20.5 hours, 1 mL of base oil (2 vol.% of Estegreen (Chevron) in microbial culture medium) was added to each flask and the flasks were incubated at 35° C. at 300 rpm. After approximately 15 minutes, an aliquot was taken from each flask and the formation of the sac-like structures and visibility of the oil soluble dye was observed using a microscope. Although the sac-like structures were slightly more agglomerated in the flask shaken at 300 rpm than the flask shaken at 150 rpm, the difference was not significant.

### Effect of Media Type on Formation of Sac-Like Structures

The effect of different types of media on the formation of sac-like structures was assessed. Approximately two loopfuls of G. sihwensis strain ATCC PTA-9635 (which is approximately 20,000 CFU to approximately 5,000,000 CFU) were inoculated into four flasks and each flask was incubated at 35° C. at 150 rpm. One flask contained 50 mL of nutrient broth (EMD Chemicals; Gibbstown, N.J.), another flask contained 50 mL of TSB (EMD Chemicals; Gibbstown, N.J.), another flask contained 50 mL of 50/50 TSB/enhanced Inakollu mineral media (Hung and Shreve (2004), “Biosurfactant Enhancement of Microbial Degradation of Various Structural Classes of Hydrocarbon in Mixed Waste Systems”, Environ. Engineering Science 21(4): 463-469; see Table 1 below for the formula of Inakollu Media and Enhanced Inakollu Media), and the fourth flask contained 50 mL of brain heart infusion (BHI) broth. After approximately 20 hours, 1 mL of base oil (2 vol. % of Estegreen (Chevron) in microbial culture medium) and 0.02 mL of Oil Red 0 (EMD Chemicals; Gibbstown, N.J.) was added to each flask and the flasks were shaken at 35° C. at 300 rpm. After approximately 15 minutes, an aliquot of 0.5 mL was taken from each flask and the formation of the sac-like structures was observed using a microscope. Sac-like structure formation was best using BHI followed by TSB, then 50/50 TSB/enhanced Inakollu mineral media, and then nutrient broth.

TABLE 1

| | | |
|---|---|---|
| Salt | Inakollu Media | Enhanced Inakollu Media |

| | | | | |
|---|---|---|---|---|
| KH₂PO₄ | 4 | g/L | 4 | g/L |
| K₂HPO₄ | 5 | g/L | 5 | g/L |
| NaNO₃ | 2 | g/L | 11.2 | g/L |
| NaCl | 0.5 | g/L | 0.5 | g/L |
| KCl | 0.5 | g/L | 0.5 | g/L |
| CaCl₂ | 0.025 | g/L | 0.025 | g/L |
| FeSO₄ | 0.25 | mg/L | 1.25 | mg/L |
| H₃BO₃ | 0.45 | mg/L | 2.25 | mg/L |
| ZnSO₄ 7H₂O | 0.75 | mg/L | 3.75 | mg/L |
| MnSO₄ 7 H₂O | 0.75 | mg/L | 3.75 | mg/L |

### Effect of Temperature on Formation of Sac-Like Structures

The effect of temperature on the formation of sac-like structures was assessed. Approximately two loopfuls of G. sihwensis strain ATCC PTA-9635 (which is approximately 20,000 CFU to approximately 5,000,000 CFU) were inoculated into three flasks, each flask containing 50 mL of 100% TSB, and each flask was incubated at 35° C. at 150 rpm. After approximately 20.5 hours, 1 mL of base oil (2 vol. % of Estegreen (Chevron) in microbial culture medium) was added to each flask. One flask was incubated at 31° C. at 150 rpm, another flask was incubated at 35° C. at 150 rpm, and the third flask was incubated at 40° C. at 150 rpm. After approximately 15 minutes, an aliquot of 0.5 mL was taken from each flask and the formation of the sac-like structures was observed using a microscope. Sac-like structure formation after approximately 15 minutes was best at 40° C., followed by 35° C. and then 31° C.

### Effect of pH on Formation of Sac-Like Structures

The effect of pH on the formation of sac-like structures was assessed. Approximately two loopfuls of G. sihwensis strain ATCC PTA-9635 (which is approximately 20,000 CFU to approximately 5,000,000 CFU) were inoculated into three flasks, each flask containing 50 mL of 100% TSB, pH 7 and each flask was incubated at 35° C. at 150 rpm. After approximately 20.5 hours, 1 mL of base oil (2 vol. % of Estegreen (Chevron) in microbial culture medium) was added to each flask. One flask was incubated at 35° C. at 300 rpm at pH 6 for 15 minutes, another flask was incubated at 35° C. at 300 rpm at pH 7 for 15 minutes, and the third flask was incubated at 35° C. at 300 rpm at pH 8 for 10 minutes. An aliquot of 0.5 mL was taken from each flask and the formation of the sac-like structures was observed using a microscope. Sac-like structure formation was best at pH 7, followed by pH 8 and then pH 6.

### Effect of CaCl₂ on Formation of Sac-Like Structures

The effect of CaCl₂ on the formation of sac-like structures was assessed. Approximately two loopfuls of G. sihwensis strain ATCC PTA-9635 (which is approximately 20,000 CFU to approximately 5,000,000 CFU) were inoculated into two flasks, each flask containing 50 mL of 100% TSB, pH 7 and each flask was incubated at 35° C. at 150 rpm. After approximately 20 hours, 1 mL of base oil (2 vol. % of Estegreen (Chevron) in microbial culture medium) was added to each flask and 0.2 grams of CaCl₂ was added to one of the two flasks. The flasks were incubated at 35° C. at 300 rpm for 15 minutes and then an aliquot of 0.5 mL was taken from each flask and the formation of the sac-like structures was observed using a microscope. Sac-like structures were formed in both flasks.

### Effect of Surfactant on Formation of Sac-Like Structures

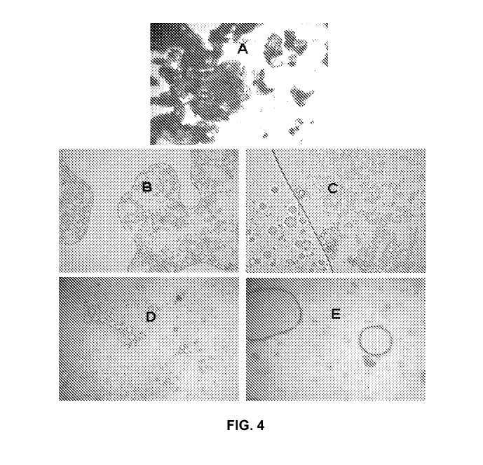

The effect of surfactant on the formation of sac-like structures was assessed. Approximately two loopfuls of G. sihwensis strain ATCC PTA-9635 (which is approximately 20,000 CFU to approximately 5,000,000 CFU) were inoculated into five flasks, each flask containing 50 mL of 100% TSB, pH 7 and each flask was incubated at 35° C. at 150 rpm. After approximately 20 hours, 1 mL of base oil (2 vol. % of Estegreen (Chevron) in microbial culture medium) was added to each flask, and no surfactant was added to one flask, 0.02% Triton® X-100 (Rohm & Haas; Philadelphia, Pa.) was added to two flasks, 0.12% Centrolex® lecithin (Central Soya; Fort Wayne, N.J.) was added to another flask, and 0.6% rhamnolipid biosurfactant (Jeneil Biosurfactant; Saukville, Wis.) was added to the fifth flask. The flasks were incubated at 35° C. at 300 rpm for 15 minutes and then an aliquot of 0.5 mL was taken from each flask and the formation of the sac-like structures was observed using a microscope. As shown in FIGS. 4B-4E, the formation of sac-like structures was adversely affected by the presence of surfactant.

### Effect of Drill Solids on Formation of Sac-Like Structures

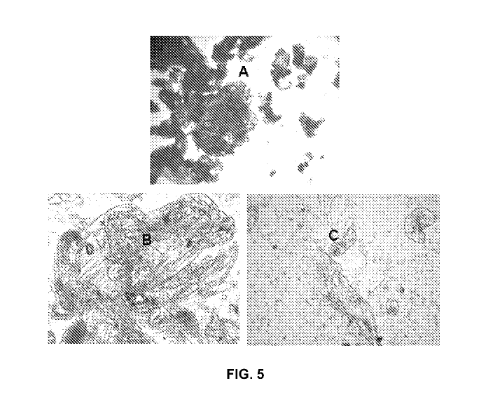

The effect of drill solids on the formation of sac-like structures was assessed. Drill solids were made from drill cuttings that were extracted with solvent to remove the drilling fluid, then sieved to a uniform particle size. Approximately two loopfuls of G. sihwensis strain ATCC PTA-9635 (which is approximately 20,000 CFU to approximately 5,000,000 CFU) were inoculated into three flasks, each flask containing 50 mL of 100% TSB, pH 7 and each flask was incubated at 35° C. at 150 rpm. After approximately 20 hours, 1 mL of base oil (2 vol. % of Estegreen (Chevron) in microbial culture medium) was added to each flask, and no drill solids was added to one flask, 5 grams of drill solids was added to another flask, and 10 grams of drill solids was added to the third flask. The flasks were incubated at 35° C. at 300 rpm for 15 minutes and then an aliquot of 0.5 mL was taken from each flask and the formation of the sac-like structures was observed using a microscope. As shown in FIGS. 5A-5C, the sac-like structures formed in the presence of the drill solids.

### Effect of Different Types of Oil on Formation of Sac-Like Structures

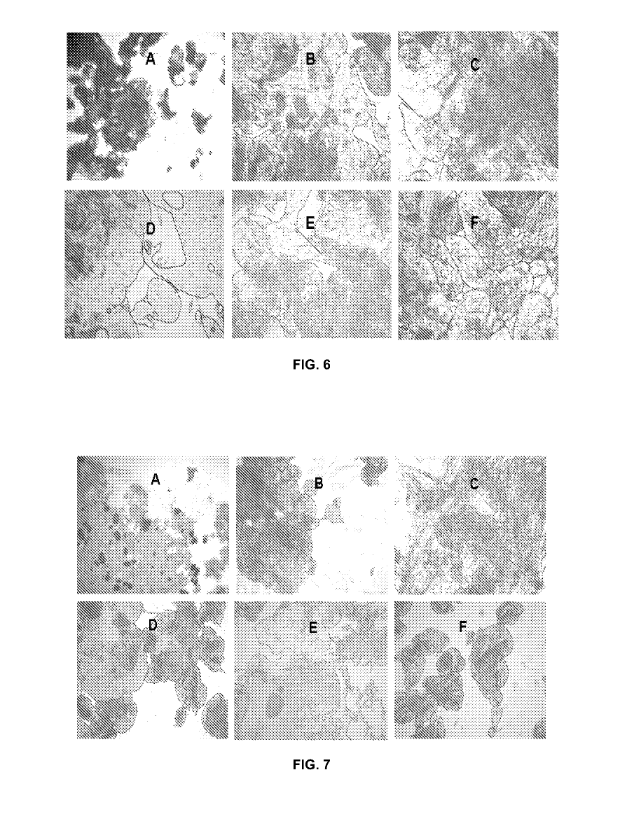

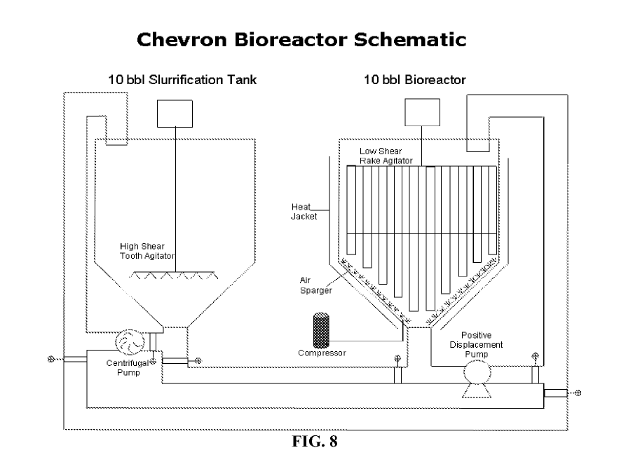

The effect of different types of oil on the formation of sac-like structures was assessed. Approximately two loopfuls of G. sihwensis strain ATCC PTA-9635 (which is approximately 20,000 CFU to approximately 5,000,000 CFU) were inoculated into six flasks, each flask containing 50 mL of 100% TSB, pH 7 and each flask was incubated at 35° C. at 150 rpm. After approximately 20 hours, 1 mL of base oil (2 vol. % Estegreen (Chevron) in microbial culture medium) was added to one flask, 1 mL of No. 2 diesel was added to another flask, 1 mL of Puredrill® IA35LV (Petro Canada; Canada) was added to another flask, 1 mL of Ametek® white oil (Ametek; Paoli, PA) was added to another flask, 1 mL of kerosene was added to another flask, and 1 mL of HDF-2000 (Total) was added to the sixth flask. The flasks were incubated at 35° C. at 300 rpm. An aliquot was taken from each flask after 15 minutes and after 1 hour, and the formation of the sac-like structures was observed using a microscope. As shown in FIGS. 6A-6F (15 minutes) and FIGS. 7A-7F (1 hour), the sac-like structures formed in the presence of all of the oils tested.

### 6.4 Biodegradation of Oil

The ability of G. sihwensis strain ATCC PTA-9635 to biodegrade oil was assessed using a total petroleum hydrocarbon (TPH) assay (EPA Method 8015B Non Halogenated Organics Using GC/FID, Revision 2, December 1996). Approximately two loopfuls of G. sihwensis strain ATCC PTA-9635 (which is approximately 20,000 CFU to approximately 5,000,000 CFU) were inoculated into six flasks containing either 50 mL of TSB or 50 mL of nutrient broth (NB). The conditions for growing the bacteria before the addition of 1% or 2% base oil (1 or 2 vol. % of Estegreen (Chevron) in microbial culture medium, respectively) and the conditions after the addition of 1% or 2% base oil (1 or 2 vol. % of Estegreen (Chevron) in microbial culture medium) are found in Table 2 below.

TABLE 2
Conditions

| | | | | | Biodegradation | | | | | | | | |
|---|---|---|---|---|---|---|---|---|---|---|---|---|---|
| | | | | | | | | | | Shake | | | |
| | | | | | | | Shake | | | 10 min. | | | |
| Growth Phase | | | | | | | 18 min, | Drain & | Cfuge & | after | | | |
| Flask | | | Temp. | Time | % | Shake | 150 rpm, | Add | Add | adding | | Temp. | Time |
| No. | Media | RPM | (° C.) | (Hrs) | Oil¹ | 10 min.² | 35° C.³ | EI⁴ | EI⁵ | EI⁶ | RPM | (° C.) | (Hrs) |
| 1 | TSB | 150 | 35 | 20 | 2 | N⁷ | Y⁸ | Y | N | NA⁹ | 300 | 35 | 20 |
| 2 | TSB | 150 | 35 | 20 | 2 | N | Y | N | N | NA | 300 | 35 | 20 |
| 3 | NB | 150 | 35 | 22 | 1 | NA | N | N | Y | N | 300 | 35 | 19 |
| 4 | NB | 150 | 35 | 22 | 1 | NA | N | N | Y | Y | 300 | 35 | 19 |
| 5 | NB | 150 | 35 | 22 | 1 | Y | N | Y | N | NA | 300 | 35 | 18.5 |
| 6 | NB | 150 | 35 | 22 | 1 | Y | N | N | N | NA | 300 | 35 | 17.5 |

¹0.5 mL of base oil (1 vol % of Estegreen in microbial culture medium) or 1 mL of base oil (2 vol. % of Estegreen in microbial culture medium) was added to the flasks.
²Flasks were shaken by hand for 10 minutes after the addition of 0.5 mL of Estegreen (Chevron).
³Flasks were shaken in a shaker oven for 18 minutes at 150 rpm at 35° C. after the addition of 1 mL of Estegreen (Chevron).
⁴After the addition of 0.5 mL of Estegreen (Chevron) and shaking by hand for 10 minutes or after the addition of 1 mL of Estegreen (Chevron) and shaking in a shaker oven for 18 minutes at 35° C., the media was drained from the flask and 50 mL of enhanced Inakollu media was added to the bacteria remaining in the flask.
⁵The contents of the flask were centrifuged, the supernatant was removed to concentrate the bacteria, and 50 mL of enhanced Inakollu media was added to the flask along with 0.5 mL of Estegreen (Chevron).
⁶The flasks were shaken by hand for 10 minutes after the contents of the flask were centrifuged, the supernatant was removed to concentrate the bacteria and 50 mL of enhanced Inakollu media was added to the flask along with 0.5 mL of Estegreen (Chevron).
⁷N means that the condition was not used.
⁸Y means that the condition was utilized.
⁹NA means not applicable and that the condition does not apply.

Approximately 17.5 to 20 hours after the addition of base oil, the contents from each flask was analyzed by TPH, and the appearance of the bacteria and media was observed. The results from the TPH analysis and the appearance of the bacteria and in the flasks are provided below in Table 3. In all of the flasks except flask number 6, the percentage of oil recovered as measured by TPH was between 49% and 57.4%. In other words, 42.6% to 51% of the total hydrocarbons present in the flasks 1 to 5 were biodegraded. In flask number 6, the percentage of oil recovered as measured by TPH was 23.3%. In other words, 76.7% of the total hydrocarbons present in flask 6 were biodegraded.

TABLE 3
Results

| | | | Size of | | | Total % |
|---|---|---|---|---|---|---|
| | Floating | | Coagulated | | State of Bacteria | Recovered |
| No. | Layer | Coagulation | Balls | Media | Under Microscope | Oil |
| 1 | Y¹ | Y | Large | Sl³ Cloudy | Bacteria agglomeration | 50.3/53.4 |
| | | (Slightly | | | | |
| | | with | | | | |
| | | swirling) | | | | |
| 2 | Y | Y | Large | Sl Cloudy | Agglomeration of sacs | 55.8 |
| 3 | Y | Y | Giant mass | Sl Cloudy | Stringy agglomeration | 57.4 |
| 4 | Y | Y | Giant ball | Sl Cloudy | Swirled agglomeration | 49 |
| 5 | Y | Y | Giant ball | Sl Cloudy | Bacteria agglomeration | 52.1 |
| 6 | N² | Y | Small | Clear | Bacteria ball | 23.3 |
| | (Sunk) | | | | | |

¹Y means that there was a floating layer in the flask.
²N means that there was no floating layer in the flask.
³Sl means slightly.

In another assay to assess the ability of the Gordonia sihwensis strain described herein to biodegrade oil, approximately two loopfuls G. sihwensis strain ATCC PTA-9635 (which is approximately 20,000 CFU to approximately 5,000,000 CFU) were inoculated into fifteen flasks containing either 50 mL of nutrient broth (NB)or 50 mL of nutrient broth and enhanced Inakollu media (1:1; NB/EI). The conditions for growing the bacteria before and after the addition of base oil are found in Table 4 below.

TABLE 4
Conditions

| | | | | | | Biodegradation Phase | | | | | |
|---|---|---|---|---|---|---|---|---|---|---|---|
| | | | | | | | | | | Initial | |
| Growth Phase | | | | | | | | | | Hand | Shaking |
| | | | Temp | | Time | % | Bacteria | | Temp | Shaking² | After 1 Hr³ |
| No. | Media | RPM | (° C.) | % Oil | (Hrs) | Oil¹ | Conc. | RPM | (° C.) | (min) | (min) |
| 1 | NB | 150 | 35 | 0 | 22 | 0.5 | 1X | 300 | 35 | 10 | 0 |
| Ctrl | | | | | | | | | | | |
| 2 | NB | 150 | 35 | 0 | 22 | 1 | 1X | 300 | 35 | 10 | 0 |
| 3 | NB | 150 | 35 | 0.3⁴ | 22 | 0.5 | 1X | 300 | 35 | 10 | 0 |
| | | | | (1pm) | | | | | | | |
| 4 | NB | 150 | 35 | 0 | 17 | 0.5 | 1X | 300 | 35 | 10 | 0 |
| 5 | NB | 150 | 35 | 0 | 22 | 0.5 | 1X | 300 | 35 | 20 | 0 |
| 6 | NB | 150 | 35 | 0 | 22 | 0.5 | 1X | 300 | 35 | 0 | 20 |
| 7 | NB/EI | 150 | 35 | 0 | 22 | 0.5 | 1X | 300 | 35 | 10 (after | 0 |
| | | | | | | | | | | 20 min) | |
| 8 | NB | 150 | 35 | 0 | 22 | Used to produce 2X bacteria conc. - | | | | | |
| | | | | | | see flask No. 9 | | | | | |
| 9 | NB | 150 | 35 | 0 | 22 | 0.5 | 2X | 300 | 35 | 10 | 0 |
| 10 | NB | 150 | 35 | 0 | 22 | 0.5 | 1X | 150 | 35 | 10 | 0 |
| 11 | NB | 150 | 35 | 0 | 22 | 0.5 | 1X | 150 | 35 | 0 | 10 |
| 12 | NB | 150 | 35 | 0 | 22 | 0.5 | 1X | 150 | 35 | 20 | 0 |
| 13 | NB/EI | 150 | 35 | 0 | 22 | 0.5 | 1X | 150 | 35 | 10 (after | 0 |
| | | | | | | | | | | 20 min) | |
| 14 | NB | 150 | 35 | 0 | 22 | 0.5 | 1X | 300 | 35 | 10 | 10 |
| 15 | NB | 150 | 35 | 0 | 22 | 0.5 | 1X | 150 | 35 | 10 | 10 |

¹1 mL of base oil (0.5 vol. % Estegreen (Chevron) in microbial culture medium) or 2 mL of base oil (1 vol. % Estegreen (Chevron) in microbial culture medium) was added to the flasks.
²Initial hand shaking means that the flask was hand shaken immediately after the addition of the base oil for the indicated time period.
³Shaking after 1 Hr means that 1 hour after the addition of the base oil, the flask was hand shaken for the indicated time period.
⁴0.15 mL of base oil (0.3 vol. % Estegreen (Chevron) in microbial culture medium) was added to the flask.

Approximately 8 hours after the addition of base oil, the contents from each flask was analyzed by TPH. Approximately 4.5 hours and 8 hours after the addition of base oil, the appearance of the bacteria and media was observed. The results from the TPH analysis and the appearance of the bacteria and media in the flasks are provided below in Table 5. In all of the flasks, the percentage of oil recovered after 8 hours as measured by TPH was between 28.8% and 51.2%. In other words, 48.8% to 71.2% of the total hydrocarbons present in the flasks were biodegraded.

TABLE 5
Results

| Results after 4.5 hours | | | | | Results after 8 hours | | | | | |
|---|---|---|---|---|---|---|---|---|---|---|
| | | | Size of | | | | Size of | | | Total % |
| | Floating | | Coagulated | Media | Floating | | Coagulated | Media | Microscopic State | Recovered |
| No. | Layer¹ | Coag.² | Ball | Clear³ | Layer¹ | Coag.² | Ball | Clear³ | of Bacteria | Oil by TPH |
| 1 | Y | N | | Y | Y | N | | Y | Loose sacs | 33.3 |
| | | | | | | (Slightly | | | | |
| | | | | | | Stringy) | | | | |
| 2 | Y | Y | | Y | N (Sunk) | Y | Small | Y | Bacteria ball | 45.8 |
| 3 | Y | N | Low | Y | Y | N | | Y | Sac material | 51.2 |
| | | | bacteria | | | (Smooth) | | | | |
| | | | amount | | | | | | | |
| 4 | Y | Y | Low | Y | N (Sunk) | Y (Low | Small | Y | Bacteria ball | 34.3 |
| | | | bacteria | | | amount) | | | | |
| | | | amount | | | | | | | |
| 5 | Y | Y | | Y | N | Y (High | Small | Y | Bacteria ball | 35.6 |
| | | | | | (almost | amount) | | | | |
| | | | | | all sunk) | | | | | |
| 6 | Y | N (Very | | Y | Y | Y | Average | Y | Sac/bacteria | 47.8 |
| | | Stringy) | | | | | | | agglomeration | |
| 7 | Y | Y | | N | N < 10% | Y | Average | Y | Sac/bacteria | 41 |
| | | | | (Slightly | floating | | | | agglomeration & | |
| | | | | Cloudy) | | | | | ball | |
| 8 | Y (sunk | Y | | Y | N (Sunk) | Y | Small | Y | Bacteria ball | 38.7 |
| | layer) | | | | | (Smooth) | | | | |
| 9 | Y | Y | | N | N < 5% | Y | Large | N (SI | Large bacteria ball | 28.8 |
| | | | | (Slightly | floating | | | Cloudy) | | |
| | | | | Cloudy) | | | | | | |
| 10 | Y | N (Slight | | N | Y | Y | Average | N (SI | Bacteria ball | 40.3 |
| | | Stringy) | | (Slightly | (mostly | | | Cloudy) | | |
| | | | | Cloudy) | floating) | | | | | |
| 11 | Y | N | | N | N < 10% | Y | Large | Y | Large bacteria ball | 35.1 |
| | | (Stringy) | | (Slightly | floating | | | | | |
| | | | | Cloudy) | | | | | | |
| 12 | Y | N | | N | Y | Y | Large | N (SI | Bacteria ball with | 48.8 |
| | | (Slightly | | (Cloudy) | | | | Cloudy) | liquid | |
| | | Stringy) | | | | | | | | |
| 13 | Y | N (Very | | Y | Y < 20% | Y | Average | Y | Bacteria ball | 44.2 |
| | | Stringy) | | | sinking | | | | | |
| 14 | Y | N | | N | Y < 10% | Y | Large | N (SI | Coagulation liquid | 49.9 |
| | | (Slightly | | (Slightly | sinking | | | Cloudy) | border | |
| | | Stringy) | | Cloudy) | | | | | | |

¹Y means that a floating layer was observed in the flask; N means that no floating layer was observed in the flask.
²Y means that coagulation was observed in the flask; N means that no coagulation was observed in the flask.
³Y means the media was clear; N means the media was not clear.

### 6.5 Bioreactor Test

A bioreactor yard test was performed to assess the ability of G. sihwensis strain ATCC PTA-9635 to biodegrade drill fluid-coated drill cuttings.

### 6.5.1 Material & Methods

### Bioreactor Assay

Seventeen gallons of a bacterial solution containing approximately 42×10⁶ CFU/mL of G. sihwensis strain ATCC PTA-9635 were added to 280 gallons of nutrient broth media (AMD Chemicals) in a 10 barrel bioreactor. G. sihwensis strain ATCC PTA-9635 was mixed in the bioreactor with a paddle mixer stirring at 25 rpm for 24 hours at a temperature maintained between 93.8° F. and 98.4°, a pH maintained between 7.0 and 7.6, and with a dissolved oxygen content maintained between 7.0 mg/L and 9.2 mg/L. After 24 hours, the 230 gallons of the bacteria/media mixture was pumped to a 10 barrel slurrification tank and immediately afterwards 774.6 lbs of oily cuttings were added to the slurrification tank. The 774.6 lbs of oily cuttings contained 616.61 lbs of dry cuttings and 158 lbs of Estegreen (Chevron) drilling mud. The drill cuttings were combined with the bacteria/media mixture in the tank and slurrified using a high shear centrifugal pump, a high shear agitator and a static mixer. The high shear centrifugal pump consisted of a 6×5×14 SPD 2.5 Mud Hog centrifugal pump complete with mechanical seal and a high shear impeller, driven by a 75 HP 460 V 60 Hz 1750 rpm explosion proof motor. The pump was run at full speed throughout the slurrification. The high speed agitator consisted of a 10 HP 460 V 60 Hz explosion proof mixer complete with high shear chopping impeller. The agitator was run at 100% of full speed during slurrification. The static shear mixer was 24″ length×4″ diameter, with 1″ steel rods with 45° offset and discharge.

After 2.5 hours, the slurry was pumped to the bioreactor where it was gently mixed with a paddle mixer at 25 rpm and recirculated with a size 60 Open Throat Auger PC pump driven by a 10 HP 460 V 60 Hz mechanical variable speed drive, operating at 10% to 25% of full speed. The maximum airflow was 55 cfu/min at 6 psi. The airflow varied depending on how many air diffusers were in operation. The slurry was recirculated back through the bioreactor using a positive displacement pump to prevent settling. The bioreactor was heated with heating tape and insulating jacket. The temperature was maintained between 95° F. and 101° F., the pH was maintained between 7.0 and 7.6, and the dissolved oxygen varied between 3.0 mg/L and 9.0 mg/L in the bioreactor. 5 kg of nutrient broth powder was added during the yard test to maintain nutrient concentrations. The fluid level remained fairly constant during the yard test because there was little evaporation, although water was added occasionally through acid additions.

See FIG. 8 for a schematic of the bioreactor and slurrification tank system. As would be appreciated by those of skill in the art, the bioreactor system may further comprise any additional components (such as lines, valves, gaskets, input conduits, output conduit, recycle loops, couplings for pH and oxygen sensors and/or for NaOH and NPK injections, etc.) needed and/or desired to optimize sequestration and/or biodegradation of hydrocarbons by the deposited bacteria, and/or to enhance the effectiveness, efficiency, speed, and/or other desirable properties achievable through use of the system. It should be noted that the system depicted in FIG. 8 is in no way intended to be limiting. The controller used can be any controller that is suitable for controlling, coordinating, manipulating, and/or optimizing the operation of one or more components of the system (such as, for example, the slurrification tank and/or the bioreactor) in a manner such that the deposited bacteria sequesters and/or biodegrades hydrocarbons. In some embodiments, the controller is a semi-automatic controller that allows that any desired degree of user input and/or control during the operation of the system. In some embodiments, the controller is an automatic controller. This bioreactor and slurrification tank system can be used with any G. sihwensis strain.

The bacterial count, pH, temperature, air flow rate, recirculation pump speed, degree of foaming, agitator speed, and dissolved oxygen were monitored during the yard test. The biodegradation of drill fluid-coated drilling cuttings was analyzed by TPH. Samples were taken for TPH analysis at various times during the yard test.

### Preparation of Drill Cuttings

Three large (20.5″ diameter×36″ length) Pierre 1 shale cores (purchased from Terratek in Utah) were broken into pieces. The Pierre 1 shale has the properties listed in Table 6.

TABLE 6

| Measurement | Value | |
|---|---|---|
| Bulk Density | 2.34 | g/cc |
| Grain Density | 2.7 | g/cc |
| Porosity | 15.8% | |
| Gas Permeability | 10⁻⁹ | md |
| UCS | 1600 | psi |
| Confined Strength (psi) | 2,500 @ 700 | |
| UC Young's Modulus | 130,000 | psi |

| | |
|---|---|
| Poisson's Ratio | 0.36 |

The Pierre 1 shale cores were crushed into simulated cuttings. Enough shale was crushed to obtain approximately 4 drums (220 gallons) of dry cuttings.

To simulate the crushing process, pieces of Pierre 1 shale were broken into cuttings-size pieces having the particle size distribution provided in Table 7.

TABLE 7

| Size (inches) | % by weight |
|---|---|
| >0.75 | 2.2 |
| 0.75-0.375 | 40.5 |
| 0.375-0.15 | 34.7 |
| 0.15-0.132 | 11.1 |
| <0.132 (fines) | 11.5 |

### Preparation of Chloride-Free Drilling Fluid Formulation

Approximately 2 drums of Estegreen-based drilling fluid formulation was prepared. To avoid toxicity to microorganisms, chlorides in the internal phase were eliminated by replacing calcium chloride with potassium formate. A chloride-free Estegreen-based formulation using potassium formate (HCOOK) is shown in the Table 8.

TABLE 8

| Component | Quantity | |
|---|---|---|
| Estegreen | 0.724 | bbl |
| Carbo-Gel | 6 | lbs/bbl |
| Omni-Mul | 8 | lbs/bbl |
| 30% HCOOK Brine | 0.187 | bbl |

| Properties | Value |
|---|---|
| Density | 8.34 lbs/gal |
| Oil/Brine Ratio | 80/20 |
| Water Phase Salinity (WPS), % HCOOK | 30.0 |
| Water Activity (Aw) | 0.80 |

Preparation of Drill Cuttings Coated with Estegreen-Based Drilling Fluid

616.6 lbs of dry drill cuttings were mixed with 18.95 gallons of Estegreen-based drilling fluid utilizing a 9 cuft capacity Stow cement mixer with a 1.5 HP electric motor. The drill cuttings and drilling fluid were mixed for approximately 30 minutes before being added to the slurrification tank.

### 6.5.2 Results

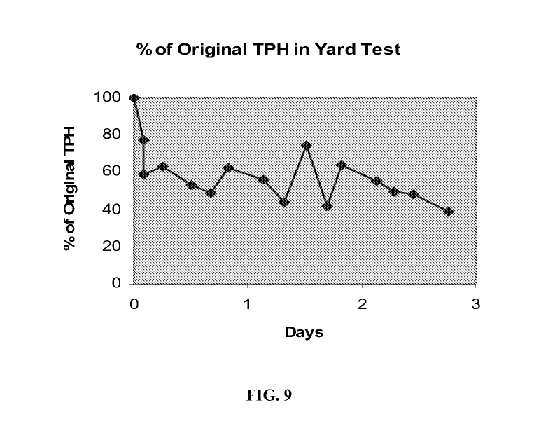

The TPH results for the bioreactor yard test are shown in Table 9 below and FIG. 9.

TABLE 9

| Time | | Percent (%) of |
|---|---|---|
| (days) | TPH | Original Recovered |
| 0 | 29,702 | 100 |
| 0.08 | 22,900 | 77.1 |
| 0.09 | 17,400 | 58.6 |
| 0.25 | 18,700 | 63 |
| 0.506 | 15,900 | 53.5 |
| 0.67 | 14,500 | 48.8 |
| 0.83 | 18,600 | 62.6 |
| 1.14 | 16,700 | 56.2 |
| 1.32 | 13,100 | 44.1 |
| 1.52 | 22,200 | 74.7 |
| 1.69 | 12,500 | 42.1 |
| 1.82 | 19,000 | 64 |
| 2.13 | 16,400 | 55.2 |
| 2.29 | 14,800 | 49.8 |
| 2.45 | 14,400 | 48.5 |
| 2.76 | 11,600 | 39.1 |

The present invention is not to be limited in scope by the specific embodiments described herein. Indeed, various modifications of the invention and their equivalents, in addition to those described herein will become apparent to those skilled in the art from the foregoing description and accompanying figures. Such modifications are intended to fall within the scope of the appended claims.

Various patents, patent applications, and publications are cited herein, the disclosures of which are incorporated by reference in their entirety and for all purposes.

### 7. FURTHER ISOLATION AND TESTING OF G. SIHWENSIS DVAD STRAINS

Various hydrocarbon sources were used to select for G. sihwensis strains that used the hydrocarbon source as a carbon source in the bacteria's metabolic pathways.

7.1 Isolation of G. sihwensis Strains

Over a two year period, various hydrocarbon sources were inoculated with a G. sihwensis culture designated DVADB01. The various hydrocarbon sources included a field biopile, linear paraffin base oil, crude oil, linear paraffin drilling mud, linear paraffin drilling mud on drill cuttings (solids), and a lab biopile. From these sources, about 200 different strains have been isolated.

### 7.2 Identification and Ribotyping

Seven of the G. sihwensis strains were chosen for speciation testing and ribotyping. G. sihwensis DVAD01 was originally isolated from the field biopile in 2007 and cryopreserved. G. sihwensis DVADC10 was isolated from the crude oil source, G. sihwensis DVADP42 from the linear paraffin base oil (an Estegreen base oil) source, and G. sihwensis strains DVADB06, DVADB07, DVADB08, and DVADB09 were isolated from the field biopile. A third party contract research organization blindly identified all seven as being G. sihwensis (each of the strains were merely identified as sample no. 1 to 7).

The seven samples were also ribotyped to determine to confirm that these were 7 different strains of G. sihwensis. The restriction enzymes EcoRI and PvuII were used to cleave the 16S and 23S rRNA genes. The DNA fragments were separated by gel electrophoresis (data not shown), and the resulting patterns were compared for similarity (Table 10).

TABLE 10

| | Similarity to | Similarity to |
|---|---|---|
| | Strain DVAD01 | Strain DVADB08 |
| Strain | (EcoRI restriction) | (PvuII restriction) |
| DVAD01 | 1.00 | 0.95 |
| DVADB09 | 0.68 | 0.58 |
| DVADB08 | 0.86 | 1.00 |
| DVADB07 | 0.81 | 0.86 |
| DVADB06 | 0.82 | 0.82 |
| DVADC10 | 0.89 | 0.96 |
| DVADP42 | 0.85 | 0.97 |

The ribotyping further confirmed multiple and different strains were being isolated from the various hydrocarbon sources.

Observations of gross morphology would also support the differentiation of the strains of G. sihwensis. Strains DVADB06, DVADB07, DVADB08, and DVADB09 had differing colony morphologies characterized as pinpoint, large, small dried, and large dried, respectively, when grown on TSA plates at 35°.

7.3 Growth of G. sihwensis

### 7.3.1 Growth in Liquid Media

G. sihwensis strains have shown the ability to grow in two liquid media: a 1) mineral media and 2) saltwater and mineral media salts (SW+MM salts). A major challenge in developing a sea water-based nutrient formulation is that nutrients must be made available for the bacteria to provide the N, P, K and trace minerals needed for bacterial growth and respiration. With sea water, this is a challenge because sea water is already saturated with NaCl, so it has very little capacity to dissolve additional salts containing the required N, P, K, Fe, etc. The specially formulated “Sea Water and Mineral Media Salts” (SW+MM Salts) provides the nutrients to the bacteria, although many of the salts are precipitated.

TABLE 11
Composition of the Mineral Media

| Salt | Concentration, ppm | |
|---|---|---|
| Na₂HPO₄ | 6 | g/L |
| KH₂PO₄ | 3 | g/L |
| NH₄Cl | 1 | g/L |
| KCl | .5 | g/L |
| FeSO₄ | 1.25 | mg/L |
| ZnSO₄ | 3.75 | mg/L |
| MnSO₄ | 3.75 | mg/L |
| Yeast Extract | .1% | w/v |
| NaNO₃ | 11.2 | g/L |

TABLE 12
Composition of the SW + MM Salts
Seawater Composition

| Ion | Conc., ppm |
|---|---|
| Cl⁻ | 19,345 |
| Na⁺ | 10,752 |
| SO₂⁻⁻ | 2,701 |
| Mg⁺⁺ | 1,295 |
| Ca⁺⁺ | 415 |
| K⁺ | 390 |
| HCO₃⁻ | 145 |
| Br⁻ | 65 |
| BO₃⁻⁻ | 27 |
| Sr⁺⁺ | 13 |
| F⁻ | 1 |

10X Mineral Media Supplement

| Salt | Concentration | |
|---|---|---|
| Na₂HPO₄ | 60 | g/L |
| KH₂PO₄ | 30 | g/L |
| NH₄Cl | 10 | g/L |
| KCl | 5 | g/L |
| FeSO₄ | 12.5 | mg/L |
| ZnSO₄ | 37.5 | mg/L |
| MnSO₄ | 37.5 | mg/L |
| Yeast Extract | 1% | w/v |
| NaNO₃ | 112 | g/L |

Concentration of Supplemental Salts
after Blending 1:9 With Seawater

| Salt | Concentration | |
|---|---|---|
| Na₂HPO₄ | 6 | g/L |
| KH₂PO₄ | 3 | g/L |
| NH₄Cl | 1 | g/L |
| KCl | .5 | g/L |
| FeSO₄ | 1.25 | g/L |
| ZnSO₄ | 3.75 | mg/L |
| MnSO₄ | 3.75 | mg/L |
| Yeast Extract | .1% | w/v |
| NaNO₃ | 11.2 | g/L |

### 7.3.2 Doubling Time

One useful feature required for rapid biodegradation of oil is rapid reproduction of bacteria. The bacteria break down the oil and use the carbon in the oil as a substrate for making new bacterial cells. 50 ml of DVADP42 liquid culture was centrifuged, the bacteria were washed twice with mineral media and resuspended in 50 ml fresh mineral media with 1.5 ml Estegreen oil added to the flask. The culture was incubated at 35° C. in a rotary shaker at 150 rpm for 3 hours. Plate counts were taken at T=0, 1.5 and 3 hours. At each time point, 300 μl were removed from the culture and further diluted (serial dilution) in mineral media. 300 μl were taken from dilutions (5, 7, 9, and 11), plated onto TSA for viable cell counts and incubated at 35° C. for 48 hours. The plate counts (CFU ml⁻¹) were recorded.

Herein, G. sihwensis strain DVADP42 were measured to grow with a doubling time of 11 minutes and 6.4 minutes in two different experiments. The rapid increase in bacterial plate counts (cfu/ml) was also indicated that complete biodegradation of the oil occurred. The doubling time was calculated from the plate counts measured during a biodegradation test. Note that the bacterial growth of strain DVADP42 (Table 13) was extremely fast between 1.5 hrs and 3 hrs (6.6×10⁹ cfu/ml to 1.98×10¹² cfu/m1). The doubling time was only 11.0 minutes.

TABLE 13

| Dilution of | Plate Count | Plate Count | Plate Count | Plate Count |
|---|---|---|---|---|
| DVADP42 | ON | @ T = 0 hrs | @ T = 3 hrs | @ T = 3 hrs |
| 10⁵ | TNTC | TNTC | TNTC | TNTC |
| 10⁷ | 67 | 36 | 79 | TNTC |
| 10⁹ | 0 | 0 | 2 | 64 |
| 10¹¹ | 0 | 0 | 0 | 6 |
| CFU/ml | 2.21 × 10⁹ | 1.19 × 10⁹ | 6.6 × 10⁹ | 1.98 × 10¹² |

ON = overnight
TNTC = too numerous to count

The doubling time D was calculated as follows: D=t/3.3(log(b/B) where t=time in minutes, b=bacteria count at end of interval, B=bacteria count at beginning of interval. For the experiment described above, growth between 1.5 hrs and 3 hrs, D=90/3.3(log(1.98×10¹²/6.6×10⁹)=11 minutes. Subsequent experiments were conducted where the oil was fully biodegraded in less than 60 minutes, which results calculated to a doubling time of only 6.4 minutes.

### 7.3.3 Growth and Biodegradation Following Cryopreservation

Another useful feature of the Chevron DVAD strains was their ability to retain their performance after being cryopreserved at −80° C. In the first three strains in Table 14, the Chevron DVAD strains had been subcultured for several months to improve their biodegradation performance. They were cryopreserved 2 weeks before being thawed, subcultured on mineral media, and re-tested. These subcultured strains fully biodegraded Estegreen base oil in 3 hours. After they were cryopreserved and thawed, they still biodegraded all of the Estegreen base oil in 3 hours (0% oil remaining) A DVADB01 strain that was not subcultured at all but was taken out of cryopreservation and thawed before the test had relatively poor performance with 60% of the oil remaining after 3 hours.

TABLE 14

| Strain | Estegreen Base Oil % @ T = 3 hours |
|---|---|
| DVADP42^{a} | 0% |
| DVADP13^{a} | 0% |
| DVADP15^{a} | 0% |
| DVADB01^{b} | 60% |

^{a}Subcultured for 2 weeks subsequent to thawing from cryopreservation
^{b}Not subcultured following thawing from cryopreservation

### 7.4 Biodegradation

DVAD strains were tested for their ability to biodegrade oil under different conditions.

### 7.4.1 Static Incubation in Mineral Media

A 50 ml centrifuge tube was filled with 10 ml of homogenized G. sihwensis strain DVADB01 strain, 10 ml Estegreen linear paraffin base oil, and 20 ml mineral media (described above). The centrifuge tube was capped and placed into an incubator oven at 35° C. under static conditions (i.e., no shaking) for one month. The capping resulted in nearly anaerobic conditions in the tube. During this time, a bacterial layer formed below the oil layer and gradually grew up into the oil layer to consume all of the oil. This indicates that G. sihwensis strain DVADB01 biodegrades oil under both aerobic and anaerobic conditions. This test was nearly anaerobic for two reasons: 1) the centrifuge tube was capped to prevent air from entering, and 2) the bacteria were located below the oil layer which prevented oxygen in the tube from contacting the bacteria.

### 7.4.2 Shaking Incubation in Mineral Media

In a shake flask test, 50 ml of mineral media containing 1.5 ml (3 vol %) of Estegreen base oil was inoculated with 2×10⁹ G. sihwensis strain DVADB01. The cap on the flask was sealed to prevent air from entering the flask. Despite the inability of air to enter the shake flask during the test, the Estegreen base oil was fully biodegraded within 6 hours. During this 6 hour incubation at [35° C.], the number of G. sihwensis strain DVADB01 increased from 2×10⁹ to 8×10¹³.

### 7.4.3 Incubation and Biodegradation in Natural Seawater

A pipette was used to transfer G. sihwensis strain DVADP42 in an unrelated experiment. Instead of disposing of the pipette, this pipette was then used to transfer crude oil into natural seawater in a shake flask. Thus, the pipette was contaminated with only minimal G. sihwensis strain DVADP42. The seawater was natural and did not contain supplemental nutrients. The flask was incubated at 40° C. for 3 days while shaking (300 rpm). After 3 days of incubation, the seawater was full of G. sihwensis strain DVADP42, and the crude oil layer was not present. Thus, the G. sihwensis strain DVADP42 remaining in a pipette following a pipette delivery had grown and biodegraded crude oil in natural seawater.

Seeding with low concentrations of G. sihwensis may have important commercial applications. For instance, G. sihwensis can be used in seawater at low concentrations to remediate crude oil spills. The above experiment demonstrates that G. sihwensis can grow and degrade oil in natural seawater without supplemental nutrients. This indicates that these results would be replicable in a natural seawater setting as well. Further, G. sihwensis could be seeded into drilling mud before discharging drill cuttings in the ocean. Thereby, the drilling mud should be biodegraded by G. sihwensis on the ocean floor.

### 7.4.4 Sac-Like Formation

G. sihwensis strain DVADB01 was incubated in tryptic soy broth for 22 hours at 35° C. while shaking (150 rpm). After 22 hours, base oil and oil soluble dye were added to solution (t=0) while continuing to incubate. The solution was sampled at multiple time points between zero and 60 minutes to observe interactions between the G. sihwensis strain DVADB01 and the base oil. Sac-like formation between G. sihwensis strain DVADB01 and the base oil was observed (via a light microscope at 1000× magnification) similar to the observations described above in the examples using G. sihwensis strain ATCC PTA-9635. At approximately 2 minutes, sacs began to form and surround the oil. Sac formation was nearly complete after about 5 minutes. At 13 minutes, the sacs appeared more highly structured. The further structure to the sacs may be due to continued complexing between the oil and extracellular polysaccharides. An extensive network of sacs was observed at 30 minutes. Some of the sacs appeared stretched and/or collapsed. This could be an artifact of the preparation of the microscope slide. Alternatively, the stretching and collapsing of sacs could be due to the agitation from the shaking. Further, the sacs could be stretching and collapsing after degradation of the oil that the sacs surround. At one hour after the base oil was added, an agglomeration of sacs was observed. However, only scarce amounts of free base oil were observed after one hour.

### 7.4.5 Biodegradation Testing

Biodegradation performance was tested. Depending on the performance achieved in the tests, subculturing conditions and acclamation conditions can be changed to achieve better biodegradation results. The test consisted of spiking an Estegreen linear paraffin base oil or crude oil into a shake flask containing media, then incubating the shake flask in the shaker oven at a prescribed shaking condition (e.g., 35° C. and 150 rpm). After 60 minutes, the shake flask was removed, and the oil biodegradation was determined

### 7.4.5.1 Estegreen Oil Biodegradation Testing

With the Estegreen oil biodegradation tests, completion of oil biodegradation was indicated by no free oil after a 25 minute centrifugation at 4400 rpm. With crude oil biodegradation tests, completion of oil biodegradation was indicated by no free oil in the flask and no color or odor of crude oil remaining in the flask. While this biodegradation completion indicator was used to measure full biodegradation, partial biodegradation could also be measured in the Estegreen tests by the amount of free oil remaining after centrifugation. For the crude oil tests, partial biodegradation was noted by the presence of visible crude oil, odor, dark coloring, bacteria agglomeration, or bacteria floating on the surface. Attempts were made to measure partial degradation of crude oil via TPH with a Turner Designs portable UV meter and Wilkes Infracal portable IR meter. However, neither portable meter was successful in measuring TPH because the background responses were high, i.e., it was difficult to distinguish a background response from the bacteria from unbiodegraded oil.

In the Estegreen biodegradation tests, nine different subcultures of G. sihwensis strains were tested. All of the strains were subcultured on Estegreen base oil in mineral media, except for subculture #4 which was subcultured on Estegreen base oil in seawater/MM salts. Each strain was diluted 1:9 in MM containing 3% Estegreen in 50 ml liquid volume, and incubating overnight at 200 rpm and 37° C. Twenty minutes prior to spiking with Estegreen base oil, the test flask was warmed at 40° C. while shaking (350 rpm). Measurements were performed 60 minutes after the flasks were spiked with 1.5% Estegreen base oil.

The results from the Estegreen biodegradation test are shown in Table 15. Complete biodegradation was achieved by G. sihwensis strains DVADP72 and DVADP69, which showed heavy bacterial growth in the flasks with no visible oil after centrifugation. Additionally, there was less than 5% oil remaining after the incubation with G. sihwensis strains DVADP71 and DVADP67. The least amount of degraded oil remained after incubation with G. sihwensis strains DVADP66 and DVADP68 and still 50% of the oil was degraded in an hour.

TABLE 15

| G. sihwensis | % Oil Remaining After |
|---|---|
| Strain | 60 min incubation |
| DVADP71 | <5 |
| DVADP68 | 50 |
| DVADP67 | <5 |
| DVADP66 | 50 |
| DVADP70 | 10 |
| DVADP72 | 0 |
| DVADP73 | 20 |
| DVADP64 | 20 |
| DVADP69 | 0 |

### 7.4.5.2 Crude Oil Biodegradation Testing

Various G. sihwensis strains were compared in a crude oil biodegradation test. 7,500 ppm of Crude 2 (Rangley County Tank #104 API 33o Chevron Crude) was biodegraded for 6 hours in a shake flask containing seawater and mineral media salts. The biodegradation test was conducted after 2 days of acclamation on seawater/MM salts and 15,000 ppm Estegreen oil, and 1 day of acclamation on 7500 ppm Crude 2 (first 5 hours) and 7,500 ppm Estegreen oil (last 18 hours). Biodegradation of Crude 2 was tested by spiking (7500 ppm) in 50 ml of a 1:4 dilution of SW/MM and incubating at 41° C. while shaking at 400 rpm.

All of the crude oil was degraded by G. sihwensis strains DVADP65, DVADP69, and DVADP71 after 6 hours. Complete degradation was indicated by no visible crude oil, no crude oil smell, and no dark coloring from asphaltenes. After incubation with G. sihwensis strains DVADP67, DVADP72, and DVADP73, there was poor bacterial growth, brown coloring, and distinctive crude oil odor. Further, G. sihwensis strains DVADP70 and DVADC25 only produced a moderate degradation as there was a light crude smell and slight darkening coloration.

What is claimed is:

1. A method for sequestering hydrocarbons comprising contacting a hydrocarbon composition with Gordonia sihwensis or a composition comprising media conditioned by Gordonia sihwensis.

2. A method for biodegrading hydrocarbons comprising contacting a hydrocarbon composition with Gordonia sihwensis or a composition comprising media conditioned by Gordonia sihwensis.

3. A method for sequestering and biodegrading hydrocarbons comprising contacting a hydrocarbon composition with Gordonia sihwensis or a composition comprising media conditioned by Gordonia sihwensis.

4. The method of claim 1, wherein the-hydrocarbon composition comprises an alkane, alkene, alkyne, cycloalkane, aromatic hydrocarbon, or a combination thereof.

5. The method of claim 2, wherein the-hydrocarbon composition comprises an alkane, alkene, alkyne, cycloalkane, aromatic hydrocarbon, or a combination thereof.

6. The method of claim 3, wherein hydrocarbon composition comprises an alkane, alkene, alkyne, cycloalkane, aromatic hydrocarbon, and or a combination thereof.

7. The method of claim 1, wherein the hydrocarbon composition comprises drill cuttings coated with drilling fluid.

8. The method of claim 2, wherein the hydrocarbon composition comprises drill cuttings coated with drilling fluid.

9. The method of claim 3, wherein the hydrocarbon composition comprises drill cuttings coated with drilling fluid.

10. A method for sequestering hydrocarbons comprising contacting a hydrocarbon composition with a composition comprising media conditioned by Gordonia sihwensis.

12. The method of claim 1, wherein the G. sihwensis is at least two different strains of G. sihwensis.

13. The method of claim 2, wherein the G. sihwensis is at least two different strains of G. sihwensis.

14. The method of claim 3, wherein the G. sihwensis is at least two different strains of G. sihwensis.

15. A method of growing Gordonia sihwensis comprising culturing Gordonia sihwensis in Sea Water and Mineral Media Salts media.

16. A method of bioremediating a crude oil spill in seawater comprising contacting the crude oil with Gordonia sihwensis.

17. The method of claim 16, wherein the G. sihwensis is at least two different strains of G. sihwensis.
